(19) United States
(12) Patent Application Publication
(10) Pub. No.: US 2025/0228687 A1
(43) Pub. Date: Jul. 17, 2025

(54) WEARABLE DEVICE TO COUNTERACT TREMOR

(71) Applicant: Equibands Inc., Vernon Hills, IL (US)

(72) Inventor: Maanav NARULA, Highland Park, IL (US)

(21) Appl. No.: 19/022,841

(22) Filed: Jan. 15, 2025

Related U.S. Application Data
(63) Continuation of application No. PCT/US23/28292, filed on Jul. 20, 2023.
(60) Provisional application No. 63/390,963, filed on Jul. 20, 2022.

(51) Int. Cl. A61F 5/01; F16F 15/31

(52) U.S. Cl. CPC A61F 5/01; F16F 15/31

(57) ABSTRACT

Devices, systems, and methods are provided to treat tremor in a subject. Devices, systems and method may comprise a wearable based configured to be worn over at least a part of a joint of the subject; at least one flywheel-spring-damper system (FSD) comprising at least one housing configured to be coupled to the wearable base; and at least one flywheel configured to counteract tremor movements in the subject, wherein the flywheel-spring damper system is configured to be coupled to the wearable base. The tremor may be a tremor in an outer extremity, typically a hand, of a subject.

### CROSS-REFERENCE

This application is a continuation of International Patent Application No. PCT/US2023/028292, filed on Jul. 20, 2023, which claims the benefit of U.S. Provisional Application No. 63/390,963, filed on Jul. 20, 2022, each of which is incorporated herein by reference in its entirety.

### BACKGROUND

The present disclosure relates to medical devices, systems, and methods, particularly for treating tremor in outer extremities of patients, such as hand tremors.

Tremor may stem from neurological disorders such as Parkinson's Disease, Essential Tremor, and Alzheimer's Disease. They manifest as uncontrollable shaking of a body part, such as the hands, in more of more axes. For instance, they may manifest as a flexion-extension of the hand or a pronation-supination of the hand, wrist and/or forearm. Tremors may make common everyday tasks such as eating, writing and dressing difficult. They may also carry an emotional burden on the patient, reflected in high depression rates and early retirement rates amongst patients experiencing tremor. Current common methods to treat tremor include medication and deep brain stimulation. Both treatments have significant downsides including but not limited to its ability to significantly reduce tremor amplitude in most of the patient population, side effects from short or long-term use, cost and risk to patient, and invasiveness of treatment. Current wearable devices in the market are not able to pair the device's efficacy in reducing the amplitude of the tremor with the size and weight of the device. They tend to be bulky, uncomfortable to wear for extended periods, and not very effective in reducing tremor.

Patents and published patent applications that are relevant include, but are not limited to: U.S. Pat. Nos. 5,058,571, 6,458,089, 6,695,794, 6,730,049, US2018266820, and US2019059733.

### SUMMARY

Systems, devices, and methods to treat tremor of patients are disclosed herein. In particular, disclosed is a wearable device that counteracts and reduces the amplitude of hand tremors, using one or more damping mechanisms including flywheel-spring-damper systems. The wearable device may be configured to be worn on the distal forearm, hand, and/or wrist of a patient. The wearable device may include a flywheel-spring-damper system coupled to a wearable base. The amount of damping provided by these mechanisms can be adjusted by the patient or other user. The wearable device may also calibrate itself, for instance when charged or otherwise powered, to account for tremor variation during and across tremor episodes.

Aspects of the present disclosure provide apparatuses to treat tremor in an outer extremity of a subject. An exemplary apparatus may be provided with one or more tremor damping mechanisms of different types. The disclosure also relates to the following numbered clause.

Clause 1. An apparatus for treating tremors in a subject, the apparatus comprising: a wearable based configured to be worn over at least a part of a joint of the subject; at least one flywheel-spring-damper system (FSD) comprising: at least one housing configured to be coupled to the wearable base; and at least one flywheel configured to counteract tremor movements in the subject, wherein the flywheel-spring damper system is configured to be coupled to the wearable base.

Clause 2. The apparatus of claim 1, wherein the wearable base is configured to be worn over at least a joint of an outer extremity of the subject, and
wherein the at least one flywheel is configured to counteract tremor movements in the outer extremity of the subject.

Clause 3. The apparatus of claim 2, wherein the outer extremity is a hand of the subject, and wherein the wearable base is configured to be worn over a wrist and/or at least a portion of the hand of the subject.

Clause 4. The apparatus of any one of claims 1 to 3, wherein the FSD system further comprises a spring system comprising at least one spring element configured to be directly or indirectly coupled to the at least one flywheel and subject.

Clause 5. The apparatus of claim 4, wherein the at least one spring element comprises at least a first spring element and a second spring element.

Clause 6. The apparatus of any one of claims 4 to 5, wherein the at least one spring element comprises one or more coil springs, gas springs, hydraulic springs and/or magnetic springs.

Clause 7. The apparatus of any one of claims 1 to 6, further comprising a mechanical advantage mechanism configured to be coupled to the flywheel.

Clause 8. The apparatus of claim 7, wherein the mechanical advantage mechanism is configured to amplify a rotational motion of the at least one flywheel.

Clause 9. The apparatus of any one of claim 7 or 8, wherein the mechanical advantage mechanism is configured to increase its effective mass to better counteract a patient's tremor.

Clause 10. The apparatus of any one of claims 7 to 9, wherein the mechanical advantage mechanism comprises one or multiple gear and pinion systems; cycloidal drives; pulley-belt systems; friction wheel systems; cable-driven joints; CAM mechanisms, continuous variable transmission mechanisms; gear trains; worm drives; chain drives; belt drives; rack and pinion mechanisms; ball screw mechanisms; roller screw mechanisms; pulleys; planetary gear systems; hydraulic systems; slider-crank mechanisms; multiple wheel and axle systems or any combination thereof.

Clause 11. The apparatus of any one of claims 1 to 10, further comprising at least one rack and pinion system and/or ball-screw system configured to increase the effective mass of the flywheel.

Clause 12. The apparatus of any one of claims 1 to 11, wherein one or multiple force transmission systems are configured to transmit a force and/or motion of the tremor movements to the flywheel system and receive a force and motion transmitted from the flywheel.

Clause 13. The apparatus of claim 12, wherein the one or multiple force transmission system comprises at least one primary force transmission system and/or at least one intermediary force transmission system.

Clause 14. The apparatus of claim 12, wherein the one or multiple force transmission system comprises at least one rack and pinion system, ball-screw system, cable-driven joint system, belt-pulley system, scotch-yoke, slider-crank, CAM-follower, cylindrical-CAM mechanisms, and/or scissor mechanism.

Clause 15. The apparatus of any one of claims 12 to 14, wherein the force transmission system comprises a fluid and/or magnetic force transmission system.

Clause 16. The apparatus of any one of claims 7 to 15, wherein one or multiple secondary mechanical advantage mechanism are configured to be coupled to at least one force transmission system and amplify the tremor movements transmitted by the outer extremity to the flywheel.

Clause 17. The apparatus of any one of claims 7 to 16, wherein at least one of the mechanical advantage mechanism or the secondary mechanical advantage mechanisms comprises at least one rack and pinion system, ball-screw system, cable-driven joint system, belt-pulley system, scissor mechanism, one or multiple gear and pinion systems; cycloidal drives; pulley-belt systems; friction wheel systems; cable-driven joints; CAM mechanisms, continuous variable transmission mechanisms; worm drives; chain drives; belt drives; rack and pinion mechanisms; ball screw mechanisms; roller screw mechanisms; pulleys; gear trains; planetary gear systems; hydraulic systems; slider-crank mechanisms; multiple wheel and axle systems or any combination thereof.

Clause 18. The apparatus of any one of claims 7 to 17, wherein the primary force transmission system comprises a biased contact mechanism configured to attach the wearable base to the subject.

Clause 19. The apparatus of any one of claims 7 to 18, wherein the primary force transmission system mechanism is configured to attach the wearable base to an outer extremity of the subject and transmit motion from the outer extremity to the flywheel-spring-damper system.

Clause 20. The apparatus of any one of claims 4 to 19, wherein the flywheel-spring-damper system further comprises an adjustment element configured to adjust a spring constant of the at least one spring element.

Clause 21. The apparatus of claim 20, wherein the adjustment element comprises:
a first adjustment element configured to adjust a spring constant of a first spring element; and
a second adjustment element configured to adjust a spring constant of a second spring element.

Clause 22. The apparatus of claim 21, wherein the first and second adjustment elements are configured to restrict or release active spring coils or engage or disengage the at least one spring elements.

Clause 23. The apparatus of any one of claims 20 to 22, wherein the adjustment element is configured to change a gas or fluid pressure of gas or fluid springs.

Clause 24. An apparatus of any one of claims 20 to 22, wherein the adjustment element is configured to adjust a magnetic field strength and/or direction in a magnetic spring system.

Clause 25. An apparatus of claim 24, wherein the FSD system is configured to store energy generated by the magnetic spring system and use the store energy to power one or multiple components of the FSD system.

Clause 26. An apparatus of any one of claims 20 to 22, wherein the adjustment element is configured to adjust a fluid bulk modulus, fluid volume, hydraulic pressure, or effective cross-sectional area of a hydraulic spring system.

Clause 27. The apparatus of any one of claims 7 to 26, further comprising a mechanical advantage adjustment element configured to change an effective mass of the flywheel.

Clause 28. The apparatus of claim 27, wherein the mechanical advantage adjustment element is configured adjust an effective mass of the flywheel by changing an extent of the applied mechanical advantage.

Clause 29. The apparatus of any one of claims 23 to 28, wherein the mechanical adjustment element is configured to adjust a gear ratio or drive ratio of the mechanical advantage system applied to the flywheel.

Clause 30. The apparatus of any one of claims 23 to 29, wherein the mechanical adjustment element comprises one or multiple gear and pinion systems; cycloidal drives; pulley-belt systems; friction wheel systems; cable-driven joints; CAM mechanisms, continuous variable transmission mechanisms; worm drives; chain drives; belt drives; rack and pinion mechanisms; gear trains; ball screw mechanisms; roller screw mechanisms; pulleys; planetary gear systems; hydraulic systems; slider-crank mechanisms; multiple wheel and axle systems or any combination thereof.

Clause 31. The apparatus of any one of claims 23 to 30, wherein the flywheel-spring-damper system further comprises an adjustment element configured to adjust a damping constant of the FSD system.

Clause 32. The apparatus of any one of claims 1 to 31, further comprising a fluid damper configured to be within the FSD system.

Clause 33. The apparatus of claim 32, wherein the fluid damper is configured to adjust the damping constant of the FSD system by increasing or decreasing at least one of a volume, pressure, viscosity, or fluid type of the fluid damper during a force transmission.

Clause 34. The apparatus of any one of claims 1 to 33, wherein an internal air resistance or internal friction within the FSD system is configured to damp the tremor movements of the subject.

Clause 35. The apparatus of any one of claims 1 to 34, comprising electromechanical and/or hydraulic mechanisms configured to control and adjust an internal friction within the FSD system.

Clause 36. The apparatus of any one of claims 1 to 35 wherein a magnetic field strength and/or distribution of an electromagnetic system may be adjusted to change the damping constant in the FSD system.

Clause 37. The apparatus of any one of claims 1 to 36, further comprising a second wearable base configured to be worn over at least part of the joint of the subject different than the first wearable base.

Clause 38. The apparatus of claim 37, wherein the second wearable base comprises a second FSD system.

Clause 39. The apparatus of any one of claims 1 to 38, further comprising a secondary flywheel configured to function in parallel or in series with the at least one flywheel.

Clause 40. The apparatus of any one of claims 1 to 39, further comprising at least one sensor configured to measure and transmit movement information of the subject;
and a software module configured to receive the movement information measured by the at least one sensor and record and analyze the measured movement information.

Clause 41. The apparatus of claim 40, wherein the movement information comprises one or more of frequency, amplitude, or timings of tremors of the subject.

Clause 42. The apparatus of any one of claims 40 to 41, wherein the software module is configured to analyze the measured movement information by differentiating between tremor movement and other movements of the subject.

Clause 43. The apparatus of any one of claims 40 to 42, wherein the software module is configured to control one or more adjustment mechanisms configured to adjust a spring constant of the spring elements in response to the measured tremor information.

Clause 44. The apparatus of any one of claims 40 to 43, wherein the software module is configured control one or more adjustment mechanisms configured to adjust an effective mass of the flywheel in response to the measured tremor information.

Clause 45. The apparatus of any one of claims 40 to 43, wherein the software module is configured to analyze the movement information over time to identify trends or changes in the subject's tremors and communicate the analysis to a remote device.

Clause 46. The apparatus of any one of claims 40 to 43, wherein the software module is configured to initiate a calibration phase when the apparatus is worn by the subject.

Clause 47. The apparatus of claim 46, wherein a controller is configured to automatically calibrate one or more settings of the FSD system to optimize damping of the subject's tremor movements.

Clause 48. The apparatus of claim 45, wherein the remote device may comprise a wearable smart device, a smartphone, a computer, a remote controller, or a smart tablet.

Clause 49. The apparatus of any one of claims 1 to 48, wherein a spring constant of the FSD system and/or an effective mass of the flywheel is configured to be manually adjusted by the subject.

Clause 50. The apparatus of claim 49, wherein a coil restrictor mechanism is configured to be manually adjusted to adjust the spring constant.

Clause 51. A system for treating tremors in a subject, comprising the apparatus of any of claims 1-48, further comprising one or multiple tuned mass damper systems.

Clause 52. The system of claim 51, wherein the one or multiple tuned mass damper systems are configured to be used in parallel or in series with the FSD system.

Clause 53. A method of treating tremors in a subject, the method comprising providing an apparatus as in any one of claims 1 to 52.

Clause 54. A method of treating tremors in a subject, the method comprising coupling an apparatus as in any one of claims 1 to 52 to an extremity of the subject.

Clause 55. The method of claim 53 or 54 further comprising entering a calibration phase when the apparatus is worn by the subject, wherein the calibration phase comprises; sensing and recording a first set of movement information of the subject; and adjusting one or more elements of the FSD system to most effectively damp a user's tremor based on the first set of movements data.

The FSD system(s), as described herein, may be removably attached to the wearable base, and a plurality of The FSD system(s) may be removably attached to the wearable base. Tremor parameters such as amplitude, intensity, and frequency may be tracked by the device and synced with a mobile and/or computer application. Information such as changes in amplitude and/or frequency of the tremor may be valuable to the user and their physicians. This data, for instance, may provide insight to the progression of the user's condition and/or if the type or dosage of medication needs to be changed.

One or more characteristics of the tremor in the outer extremity of the subject may be measured and recorded, for example the amplitude and frequency of the tremor(s). The measurement may be performed by a mobile and/or computer application coupled to the apparatus.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a side perspective view of an uncovered device that counteracts and reduces tremor worn over a wrist and hand of a subject, in accordance with some embodiments described herein;

FIG. 2A1 shows a perspective view of a flywheel-spring-damper system employing a cable-driven joint mechanism in accordance with some embodiments described herein;

FIG. 2A2 shows a perspective view of a section of a flywheel-spring-damper system worn over a wrist, in accordance with some embodiments described herein;

FIG. 2A3 shows a side view of a section of a flywheel-spring-damper system worn over a wrist, in accordance with some embodiments described herein;

FIG. 2A4 shows a top-down view of a section of a flywheel-spring-damper system worn over a wrist, in accordance with some embodiments described herein;

FIG. 2A5 shows an exploded view of a section of a flywheel-spring-damper system worn over a wrist, in accordance with some embodiments described herein.

FIG. 3A1 shows a perspective view of a flywheel-spring-damper system in accordance with some embodiments described herein;

FIG. 3A2 shows a top-down view of a section of a flywheel-spring-damper system worn over a wrist, in accordance with some embodiments described herein;

FIG. 4 shows a perspective view of a flywheel-spring-damper system employing a cable-driven joint mechanism and pulley-belt system with torsional springs;

FIG. 5 shows a hand tremor amplitude graph of a user experiencing a tremor wearing different treatment devices;

FIG. 6 shows a hand tremor amplitude graph of a user experiencing a tremor wearing different flywheel-spring-damper systems;

FIG. 7 shows a hand tremor amplitude graph of a user experiencing a tremor wearing flywheel-spring-damper systems having different effective mass;

FIG. 8A1 shows a top-down view of a flywheel-spring-damper system with a continuously variable transmission system, in accordance with some embodiments described herein;

FIG. 8A2 shows a top-down view of a continuously variable transmission system, in accordance with some embodiments described herein;

FIG. 8A3 shows a side view of a continuously variable transmission system, in accordance with some embodiments described herein;

FIG. 9A1 shows a perspective view of a flywheel-spring-damper system employing a lever-based cable-driven force transmission mechanism in accordance with some embodiments described herein;

FIG. 9A2 shows a side view of a flywheel-spring-damper system employing a lever-

based cable-driven force transmission mechanism in accordance with some embodiments described herein;

FIGS. 10A1-10A6 illustrate multiple configurations of a primary force transmission system employing a contact mechanism configured to be attach to the hand H in accordance with some embodiments described herein;

FIG. 11A1 shows a flow diagram of the device functions in accordance with some embodiments described herein;

FIG. 11A2 shows a flow diagram of the device functions in accordance with some embodiments described herein;

FIG. 12 illustrates a flywheel-spring-damper system employing a ball-screw system in accordance with some embodiments described herein;

FIG. 13 shows a computer system that is programmed or otherwise configured to implement methods provided herein;

FIG. 14 illustrates a magnet and rack configuration of a rack and pinion system in accordance with some embodiments described herein.

### DETAILED DESCRIPTION OF THE INVENTION

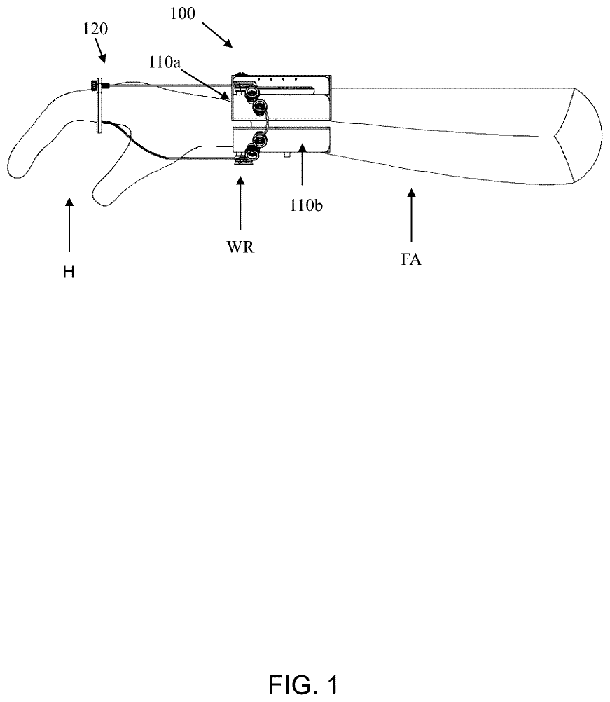

Disclosed herein are systems, devices, and methods to treat tremor in outer extremities by counteracting and reducing tremor movement(s) of the outer extremities. Referring to FIG. 1, a non-invasive, wearable device 100 that reduces tremor may comprise a wearable base 110 with flywheel-spring-damper (FSD) system(s) 200. The wearable base 110 may be worn over at least a portion of the hand H of the user and/or at least a portion of the wrist WR or forearm FA of the user. The FSD system(s) 200 may be coupled to the proximal portion of the wearable base 110. The FSD system(s) 200 may be placed above and/or below the user's wrist WR and/or distal forearm FA. Alternatively, or in combination, the FSD system(s) 200 may be placed on either lateral side (i.e., left and/or right sides) of the user's wrist WR and/or distal forearm FA. In some embodiments, the FSD system(s) 200 are detachable, such that the user can choose to wear one or multiple FSD-contained devices 100. One or more FSD-contained devices 100 may be placed above, below, left of, right of, and/or or otherwise around the user's wrist WR and distal forearm FA. In some embodiments, a plurality of FSD-contained devices 100 may be worn around the user's wrist WR and/or distal forearm FA, as in a bracelet. At least some, if not all, of the FSD devices 100 may be curved to accommodate for the shape of the user's wrist WR and/or distal forearm FA.

The FSD-contained devices 100 may comprise one or multiple systems, and one or multiple components within those systems. The systems may comprise one or multiple flywheel-spring-damper systems 200, flywheel-mechanical advantage mechanisms 400, spring systems 220, force transfer systems 120 (i.e., primary force transfer systems 120a and intermediary force transfer systems 120b), secondary mechanical advantage systems, frequency adjustment systems, and/or damping systems.

### Flywheel-Spring and Flywheel-Spring-Damper Systems

The flywheel-spring or flywheel-spring-damper (FSD) system 200 may comprise at least one flywheel 210 that may rotate in place with at least one spring element of a spring system 220 facilitating the motion between the flywheel 210 and the body part experiencing the tremor (e.g., hand H). The FSD system 200 may be rigidly attached to the wearable base 110, which may be rigidly attached to the hand H, wrist WR, and/or forearm FA via straps or other mechanisms.

The motion of the hand H due to tremor may exert a force on the spring element(s) 220 directly or via one or more force transfer systems 120 as described in S101-S104. The spring element(s) of the spring system 220 may consequently exert a force on the flywheel 210 directly or via one or more intermediary force transfer systems 120 as described in S105. The force exerted on the flywheel 210 may cause the flywheel 210 to rotate, such that it in turn exerts a force on the spring element(s) 220 directly or via the one or more force transfer systems 120. The force exerted on the spring element(s) 220 from the flywheel 210 may consequently exert a force on hand H directly or via the one or more force transfer systems 120 (i.e., a second intermediary force transfer system) as described in S106. In some embodiments a second intermediary force transfer system trigger motion in a mechanical advantage system 400 which may be configured to trigger motion of flywheel 210 as described in S106-S107. The motion of the flywheel 210 may interfere with the tremor-induced motion of the hand H and may result in the reduction in the amplitude of the tremor.

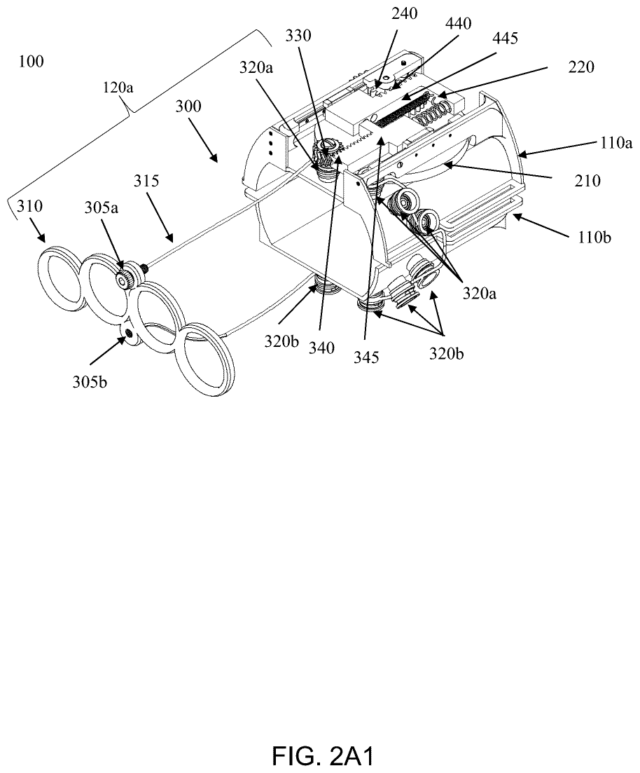

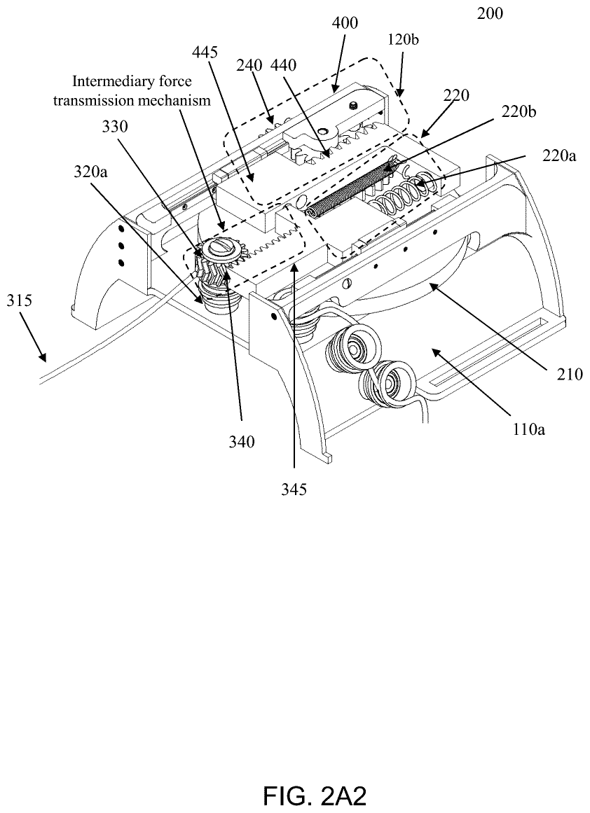

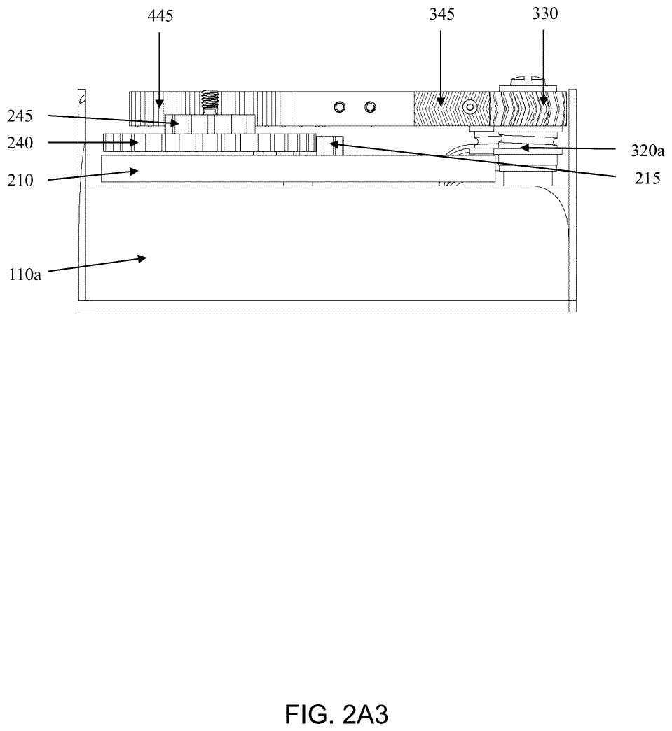

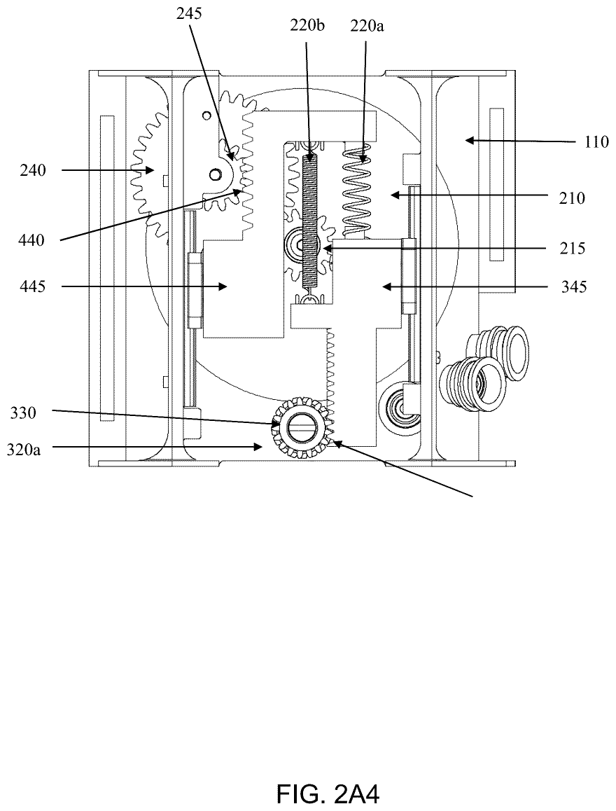

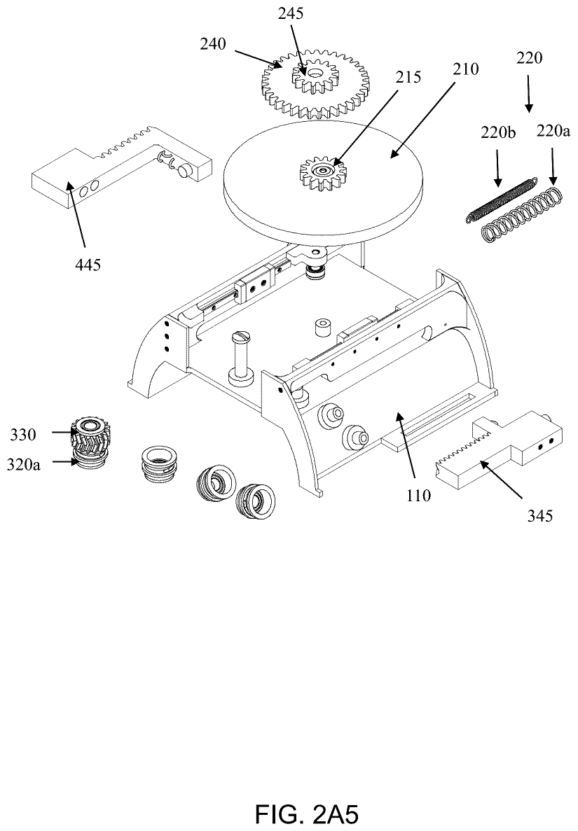

Referring to FIG. 1 and FIGS. 2A1-2A5, during a flexion-extension tremor of the hand H starting from rest, the rotational movement of the hand H upwards S101 may exert a force on the spring element(s) 220 via force transfer systems 120 as described through S102-S104, which may cause the spring element(s) 220 to exert a force on the flywheel 210 via force transfer systems 120. In this configuration, the force exerted on the flywheel 210 may cause the flywheel 210 to first rotate counter-clockwise. This flywheel rotation in turn may cause the flywheel 210 to exert a force on the spring element(s) 220 via force transfer systems 120. The spring element(s) 220 may then exert a force on the hand H via force transfer systems 120, which may interfere with and reduce the amplitude of the hand tremor as described in S105-S107.

Similarly, referring to FIG. 1 and FIGS. 2A1-2A5, a downward rotational movement of the hand H starting from rest S101 may first cause the flywheel 210 to rotate clockwise. The configuration of these systems and/or components may be altered such that an initial upward motion of the tremor may first induce a clockwise flywheel rotation, and the initial downward motion may induce a counter-clockwise flywheel rotation.

The continuous upward-downward rotational movement, or flexion-extension, of the hand H may cause a continuous clockwise-counterclockwise rotational movement of the flywheel 210. The resulting harmonic oscillations of the hand H and flywheel system 210 may be phase shifted with respect to one another. The motion of the hand H may be interfered with by the motion of the flywheel 210, and the amplitude of the hand tremor may decrease as a result of the flywheel-spring-damper system(s) 200. The degree to which the motion of the flywheel system 210 is out of phase from the motion of the hand H due to tremor may depend upon multiple variables such as the system's effective resonator (flywheel 210) mass, effective spring constant, and effective damping constant. The degree to which these motions are out of phase may impact the degree to which the amplitude of the hand decreases, though this may not be a linear relationship. For instance, this does not imply that a 180-degree out of phase flywheel system motion will most reduce the amplitude of the hand due to tremor. A 90 degree out of phase motion, for instance, may yield a greater reduction in tremor amplitude.

In the case where the user does not have the device 100 comprising an FSD system 200, the energy of the system may be defined by only the kinetic energy generated by the tremor motion of the user's hand H. In the case where the user is wearing a device comprising one or multiple FSD system(s) 200, as described herein, assuming no additional energy is input into the system, then the total energy of the system would be equal to the energy of the system not comprising the FSD system 200. In the case with FSD system(s) 200, the energy is distributed between the kinetic energy of the tremor motion of the hand H and the kinetic energy of the FSD system(s) 200. Moreover, the frequency of the tremor may not be affected by the inclusion of the FSD system(s) 200. This may imply that the kinetic energy of the tremor motion of the hand H in the case with the FSD system(s) 200 is lower than the energy of the hand H without the FSD system(s) 200. As a result, the amplitude of the hand H due to the tremor in the case with the FSD system(s) 200 may be lower than the amplitude of the hand H due to the tremor in the case without the FSD system(s) 200.

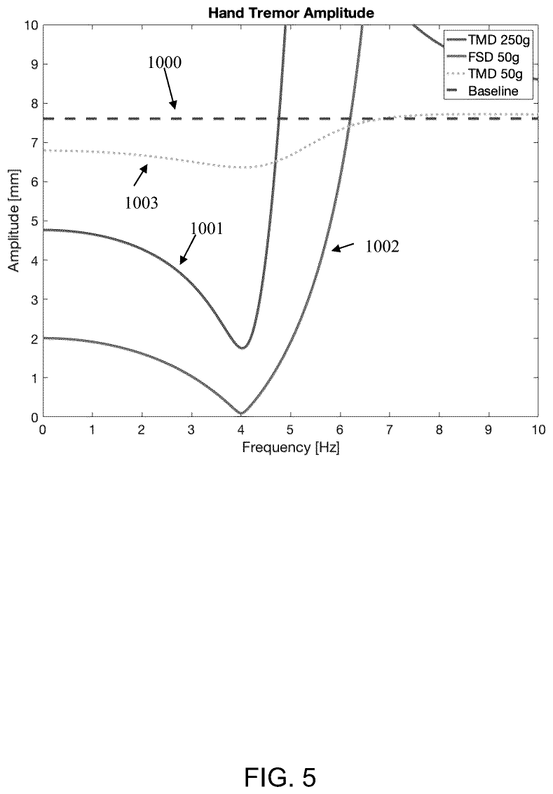

Benefits of using a flywheel-spring damper system 200, as described herein, provide more effective tremor treatment to users while reducing the weight required by the device to damp tremors compared to other mechanical or electromechanical tremor treatment devices. The reduced weight additionally may provide a more comfortable and effective treatment for tremors than other heavier or bulkier mechanical or electromechanical tremor treatment devices. The effectiveness of the lighter FSD system 200 may be seen in the graph of FIG. 5. The graph shows a user experiencing a tremor at a frequency of 4Hz with a base amplitude of 7.6 mm 1000. A tuned mass damper system with a resonator of weight 250 grams, and optimized for tremor frequencies of 4Hz, may reduce the tremor amplitude to 1.75 mm 1001. This implies a tremor reduction of ˜77%.

As shown by 1002, when all else is kept constant (e.g., the system damping constant), implementing a flywheel-spring-damper system 200 with flywheel 210 weight of 50 grams (5 times lighter than the aforementioned TMD resonator), optimized for tremor frequencies of 4Hz (by optimizing the spring constant) and having all its components fit within the same practical design constraints (e.g., size) of the TMD-contained device worn on hand H, it is reasonable to suggest that the FSD-contained device may achieve an effective mass of 1.2 kg. The effective mass of the flywheel 210 may be the mass that the flywheel 210 seems to operationally have during rotation or harmonic motion. This mass may be larger than the “rest mass” of the flywheel 210, which may be its nominal mass at rest. Given these design parameters, referring to FIG. 5., the tremor amplitude may be reduced to 0.1 mm 1002. This implies a tremor reduction of ˜98.7%. Thus, even with a significantly lower resonator weight, an FSD system 200 may be more effective in reducing the amplitude of tremor than a TMD system.

When the user isn't experiencing tremor, the user may only feel the “low weight” of the rest mass (50 grams) of the flywheel 210. During tremor, the user may experience the resistive force generated by the “higher weight” of the flywheel's 210 effective mass (up to 1.2 kg in this case).

On the other hand, assuming the same damping constant, if the TMD system had a resonator weight of 50 grams, the tremor amplitude may only be reduced to 6.36 mm 1003. This implies a tremor reduction of only 16.3%. Therefore, for the same resonator weight in this case, the FSD system 200 may provide over 83% greater tremor reduction.

In this way, the implementation of a FSD system 200 over a TMD system may provide the important benefit of being more usable and practical for the user. A low weight is one of the most important customer requirements for a tremor-reducing wearable device. The greater efficacy-to-weight ratio of an FSD system 200 over a TMD system implies that for the same level of efficacy, the FSD system 200 may employ a lower resonator weight, which may result in a lower overall device weight. A lower weight may be more comfortable for the user and may enable them to wear the device for longer periods. The higher effective mass of the FSD system 200 may be felt by a user only during a tremor, which may provide the necessary force to counteract the tremor.

The ability of an FSD system 200 to reduce tremor with a significantly lower weight as compared to a TMD system may also enable the FSD-contained device 100 to have a smaller form factor. This may provide aesthetical and practical benefits to the user. A small size is another important customer requirement for a tremor-reducing wearable device. A smaller profile may make the device sleeker and more aesthetically pleasing. It may also make the device more discreet and less noticeable, for instance, by being covered more easily under clothing. Practically, a smaller form factor may prevent the device from interfering with other objects that the user may encounter while wearing the device.

In this way, the lower weight and smaller form factor of an FSD-contained device 100 may provide significant benefits to the user over a TMD-contained device. The FSD-contained device 100 may be designed to be of lower weight, smaller size, and greater effectiveness in reducing tremor.

In some embodiments, the flywheel-spring system 200 may comprise one or multiple flywheels 210. The flywheels 210 may be arranged in parallel or in series with one another. The inclusion of flywheels 210 in parallel or in series may increase the range of tremor frequencies over which the FSD system is effective in reducing tremor by a specified amount.

The flywheel 210 may comprise high density materials such as tungsten, mercury, lead, molybdenum, steel, bismuth, copper, nickel, bronze, solder, brass, iron, steel, aluminum, manganese, tungsten carbide, tungsten steel, tungsten alloys, aluminum alloys, gold alloys, silver alloys, tin, and alloys of the above metals, to maximize its effective mass for a given volume. Increasing its effective mass may improve the FSD system 200 ability to reduce tremor.

### Mechanical Advantage Mechanism to the Flywheel.

The FSD system 200 may also comprise one or multiple mechanical advantage mechanisms that act on the flywheel 210, referred to herein as flywheel-mechanical advantage mechanisms 400. The flywheel-mechanical advantage mechanism 400 may amplify the motion of the flywheel 210. In some embodiments the flywheel-mechanical advantage mechanism 400 may increase the angular displacement and/or rotational speed of the flywheel 210 for a given input force and/or displacement. The amplified motion of the flywheel 210 may increase its effective weight. In some embodiments, the amplified motion may cause the out-of-phase motion of the flywheel 210 to have a greater impact in interfering with the hand tremor motion as compared to the scenario without this mechanical advantage. The increased rotational speed of the flywheel 210 may imply it has a greater kinetic energy than without the mechanical advantage mechanism. As no energy is added to the overall system, and more energy is allocated to the flywheel system 210 (with mechanical advantage), it may imply that less energy is allocated to the motion of the hand H, which causes a greater reduction in the amplitude of the hand tremor.

This mechanical advantage may be achieved by using gears 240 or gear trains. A small-radius pinion 215 may be attached to the flywheel 210. In some embodiments, the pinion 215 may be rigidly attached to the flywheel 210. In some further embodiments, the pinion 215 may be attached to the flywheel 210 concentrically. In non-limiting embodiments, the pinion 215 may be rigidly attached to the to the flywheel 210 concentrically. A larger gear 240 may be meshed with the pinion 215. The larger gear 240 meshed with the pinion 215 may amplify the angular speed of the pinion 215 and flywheel 210.

The rotation of the gear 240 may also be amplified separately. A secondary pinion 245 may be attached to the gear 240. In some embodiments, the secondary pinion 245 may be rigidly attached to the gear 240. In some further embodiments, the secondary pinion 245 may be attached to the gear 240 concentrically. In a non-limiting embodiments, the secondary pinion 245 may be rigidly attached to the to the gear 240 concentrically. The secondary pinion 245 may be meshed with a rack and pinion system 440, or other force transfer system 120 as described further in a later section. This may amplify the rotation of the gear 240, such that gear 240 teeth speed is greater than it would be if the gear 240 was meshed directly with the rack 445 in the rack and pinion system 440. A faster gear 240 teeth speed may imply a faster pinion 215 teeth speed and larger pinion 215 angular velocity, which may imply a larger flywheel 210 angular velocity.

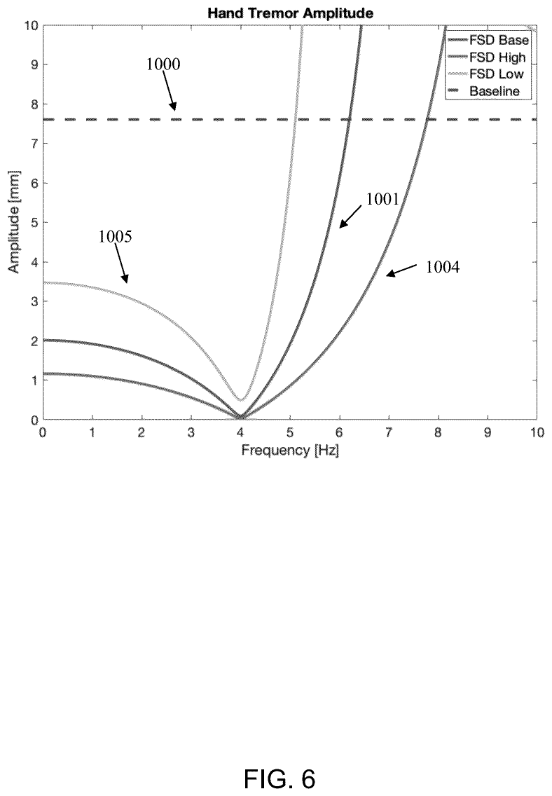

In a non-limiting example, a mechanical advantage system acting on a flywheel 210 configured to increase the effectiveness of the FSD system 200 to counteract a user's tremor. The mechanical advantage system may comprise a rack and pinion system configured such that decreasing the diameter of the pinion 215 and secondary pinion 245, and/or increasing the diameter of the gear 240, the extent of the mechanical advantage on the flywheel 210 may be increased. This may result in the flywheel 210 rotating faster and to a greater extent in response to the force exerted on it, directly or indirectly, by the hand H. This may increase the effective mass of the flywheel 210. When this configuration of the FSD system 200 is also optimized to counteract tremor of frequency 4 Hz, the extent of the tremor reduction may be increased as compared to the base case without a mechanical advantage mechanism acting on the flywheel 210. Referring to FIG. 6, the exemplary configuration describer above may reduce the amplitude of the hand tremor to 0.02 mm, shown by in “FSD High” 1004, implying a device efficacy of 99.7%. Similarly, increasing the radius of the flywheel 210 may increase the effective mass of the FSD system 200 and increase the efficacy of the device in reducing tremor.

In contrast, by increasing the diameter of the pinion 215 and secondary pinion 245, and/or decreasing the gear 240 diameter, the effect of the mechanical advantage on the flywheel 210 may be decreased. This may result in the flywheel 210 rotating slower and to a lesser degree in response to the force exerted on it, directly or indirectly, by the hand H. This may decrease the effective mass of the flywheel 210. Assuming this new configuration of the FSD system 200 is also optimized to counteract tremor of frequency 4 Hz, the extent of the tremor reduction may be decreased as compared to the base case. Referring to FIG. 6, this configuration may reduce the amplitude of the hand tremor to 0.5 mm, shown by “FSD Low” 1005, implying a device efficacy of 93.4%. Similarly, decreasing the radius of the flywheel 210 may reduce the effective mass of the FSD system and decrease the efficacy of the device in reducing tremor.

In some embodiments, multiple gears 240 and secondary pinions 245 may be used to achieve a mechanical advantage. In some embodiments, one or multiple cycloidal drives may be used to achieve this mechanical advantage. In some embodiments, one or multiple pulley-belt systems may be used to achieve this mechanical advantage (described further in Force Transfer Systems section). In some embodiments, one or multiple friction wheel systems may be used to achieve this mechanical advantage. In some embodiments, one or multiple wheel and axle systems may be used to achieve this mechanical advantage. In some embodiments one or multiple gears 240 and secondary pinions 245; cycloidal drives; pulley-belt systems; friction wheel systems; cable-driven joints; CAM mechanisms, continuous variable transmission mechanisms; worm drives; chain drives; belt drives; rack and pinion mechanisms; ball screw mechanisms; roller screw mechanisms; pulleys; planetary gear systems; hydraulic systems; slider-crank mechanisms; multiple wheel and axle systems or any combination thereof may be used to achieve this mechanical advantage.

In some embodiments, a goal of the FSD system 200 is to generate larger inertia forces for a given resonator mass. By increasing the effective mass of the flywheel 210, the FSD system 200 may further interfere with the motion of the hand H due to tremor. This may further reduce the amplitude of the hand tremor. The flywheel-mechanical advantage mechanism 400 may increase the effective weight of the flywheel 210 by amplifying its motion in response to a given input force from the hand H. The amplified, out-of-phase motion of the flywheel system 210 may thus have a greater impact in reducing the amplitude of the hand tremor.

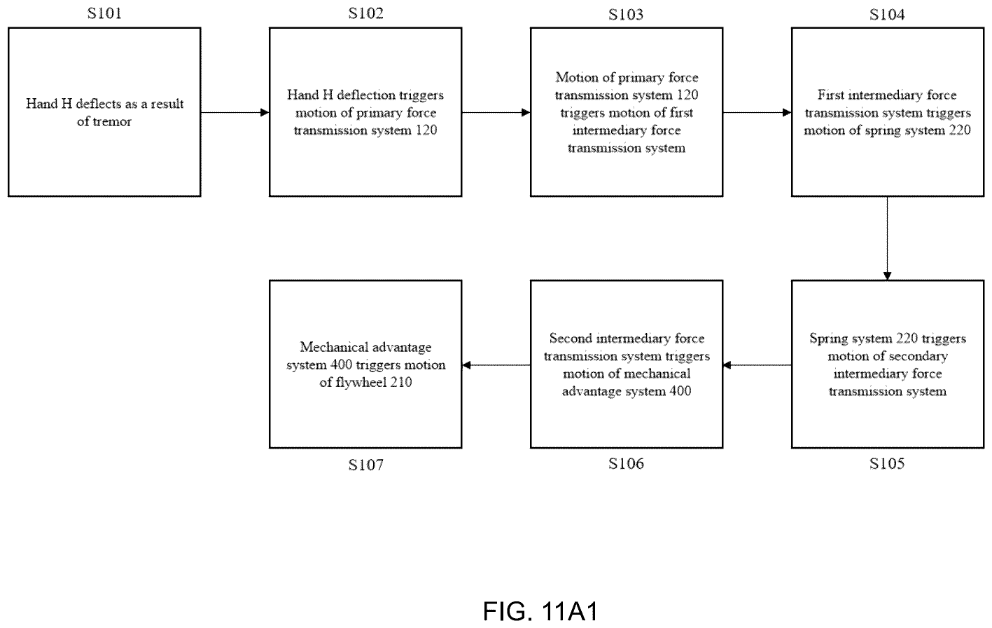

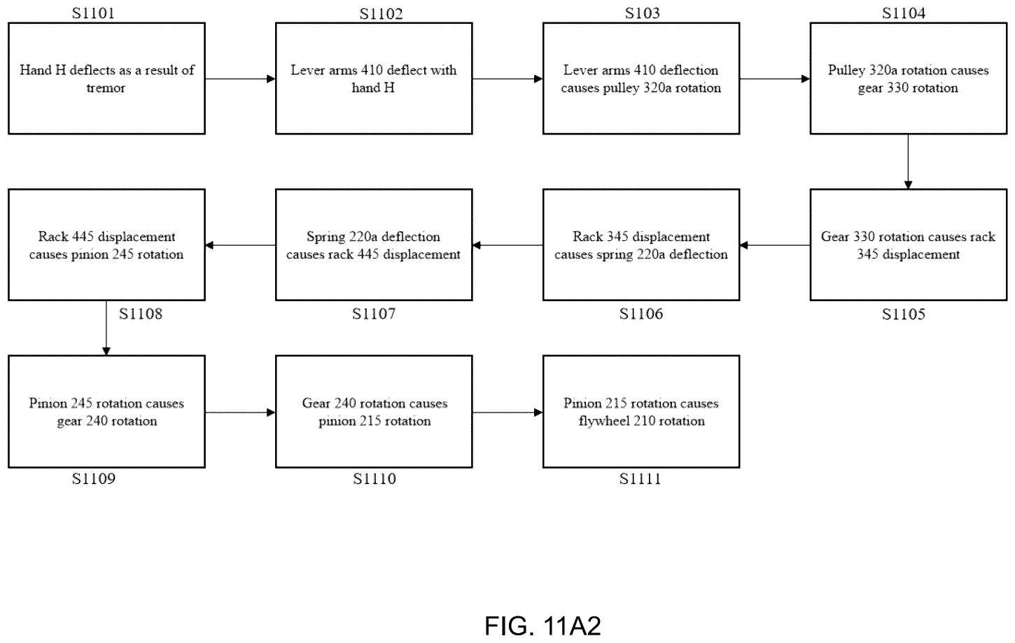

Referring to FIG. 11A1 in some embodiments, the hand H may deflects as a result of tremor S101 triggering motion of primary force transmission system 120 S102. The motion of primary force transmission system 120 may trigger motion of first intermediary force transmission system S103. The motion of the first intermediary force transmission system may trigger motion of a spring system 220 S104. The spring system 220 may then trigger motion of a secondary intermediary force transmission system S105. The motion of the second intermediary force transmission system may trigger motion of a mechanical advantage system 400 S106. The motion of the mechanical advantage system 400 may then trigger motion of the flywheel 210 S107, which may interfere with and reduce the amplitude of the hand tremor.

### Spring Systems

The spring system 220 refers to the spring element configuration in the FSD system(s) 200. The spring system 220 may comprise one or multiple spring elements 220a, 220b, or 220c which may facilitate the connection and motion between the hand and flywheel 210. The effective spring constant of the spring system 220 may be important in determining and/or influencing the motion response of the flywheel system 210 and may contribute to the out-of-phase motion of the flywheel 210 relative to the hand tremor motion. It may impact the degree to which the resulting hand amplitude is decreased relative to the baseline tremor amplitude without the FSD system(s) 200.

Referring to FIG. 2A2, the spring elements of the spring system 220 may comprise one or multiple coil springs such as traditional compression spring 220a and/or extension springs 220b. These springs 220 may be used in isolation or combination with each other. The spring element(s) 220 may be configured in parallel and/or in series with one another.

In some embodiments, one or multiple torsional springs 220c may be used in isolation or in combination with the compression springs 220a and/or extension springs 220b. In some embodiments, the one or more springs of the spring system 220 may be linear, non-linear or constant-force springs used in isolation or in combination with one another. In some embodiments the springs may include but are not limited to spiral, arc, leaf, Belleville, wave, conical, bistable, clock, variable-rate springs and or a combination thereof. In some embodiments, a combination of different spring elements within the spring system 220 may be configured such that the effective spring constant is linear (in that the force-displacement relationship is linear) or non-linear to a specified range. The one or multiple springs of the spring system 220 may be pre-tensioned and/or pre-loaded. In some embodiments only some of the multiple springs of the spring system 220 may be pre-tensioned and/or preloaded. In some embodiments, having one or multiple springs 220 be pre-tensioned and/or pre-loaded may improve the stability and/or precision of the spring elements 220.

In some embodiments, the spring elements may comprise one or multiple hydraulic systems. The hydraulic system(s) may comprise one or multiple cylinders, pistons and piston rods, compressible or incompressible fluids, pressure controls, or a combination thereof. In some embodiments, a displacement to a piston rod and piston may displace a fluid and affect a hydraulic pressure in a cylinder. In a non-limiting example, in a flexion-extension tremor, a hand displacement upwards may cause an inward displacement of a first piston and piston rod directly or via force transfer mechanism(s) 120. In the case of a largely incompressible fluid (i.e., a liquid), this displacement may generate a hydraulic pressure in the cylinder. This may exert a force on a second piston, which may directly or indirectly exert a force on the flywheel system 210. In some embodiments, the greater the displacement caused, the greater the hydraulic pressure, and the greater the force exerted on the flywheel system 210. In this way, the hydraulic system may act as spring element(s) in the spring system 220. In some embodiments, the hydraulic system may facilitate the motion and force exerted by the flywheel system 210 to the hand H. The spring system 220 may be a hydraulic spring system. The hydraulic spring system 220 may further comprise one or multiple coil springs 220a, 220b that are used in conjunction with the hydraulic system.

In some embodiments, the spring system 220 may comprise one or multiple gas springs that employ methods of using gas or fluid pressure to replicate spring behavior. In some embodiments, one or multiple bellows and/or flexible tubes (collectively called tubing) may contain a variable pressure fluid. The fluid may comprise one or multiple gases and/or fluids. Fluids may include but are not limited to air, nitrogen, oxygen, carbon dioxide, helium, argon, hydrogen, other ideal gases, compressed industrial gases, other inert gases, or a mixture or one of more of the above gases. The tubing may directly or via force transfer mechanisms 120 facilitate the motion between and apply forces to the hand H and flywheel systems 210. The tubing material and/or construction may be flexible. The flexible tubing material and/or construction may compress and expand depending on the strength and directions of the forces exerted on it. An internal fluid pressure in the tubing may influence the overall stiffness and/or effective spring constant of the spring system 220. The movement of the hand H upwards may exert a force directly or indirectly on the tubing. The force may compress the tubing. Compressing the tubing may increase the fluid pressure in the tubing. The increased pressure may result in the tubing exerting a force directly or indirectly on the flywheel system 210. The tubing may contain one or multiple internal or external coil springs. In some embodiments, the spring elements 220 may comprise one or multiple gas spring systems. The gas spring system may employ cylinders and pistons similar to the hydraulic spring system described herein.

In some embodiments, one or multiple spring systems 220 may comprise one or multiple antagonistic spring systems. In some embodiments, one or multiple antagonistic spring systems may be employed. Antagonistic spring systems may include multiple spring element(s) The multiple spring elements may comprise a one or multiple compression springs 220a and/or one or multiple extension springs 220b. In some embodiments, the compression springs 220a and the extension springs 220b may individually generate forces in opposing directions. In some further embodiments, the spring system 220 as a whole is configured to generate a net force in one direction depending on the magnitude of the forces acting on it. In some embodiments, one or multiple springs 220a, springs 220b, or a combination thereof may be pre-loaded spring. The spring system(s) 220 may further comprise fixed or flexible joints, wires with tensioning mechanisms, pulleys and gears, mechanical linkages, or a combination thereof.

In some embodiments, one or multiple spring system(s) 220 may comprise one or multiple magnetic springs. Multiple configurations may be used. In some embodiments, the one or multiple magnetic springs may comprise one or multiple electromagnets and/or permanent magnets (collectively referred to as magnets). The magnets may be placed on or between a flywheel system 210 and outer extremity experiencing tremor such as the hand H. In some embodiments, the one or multiple magnets may be placed directly and/or indirectly on the flywheel 210 and hand H. In some embodiments, the one or multiple magnets may be placed on or in one or multiple intermediary components. In some embodiments, the one or multiple magnets may be placed in a force transfer system(s) 120. In some embodiments, the one or multiple magnets may be placed on a fixed or a moving support structure. In some embodiments, the one or multiple magnets may be placed in a force transfer system(s) 120, on a fixed or a moving support structures, or a combination thereof. In some further embodiments, one or multiple magnets may be placed side by side and parallel to a direction of motion of the FSD system 200. In some further embodiments, one or multiple magnets may be stacked on top of each other and placed perpendicular to the direction of motion of the FSD system 200. The multiple magnets may be of the same or opposite polarity, or a combination thereof. In some embodiments, a magnetic field created by the one or multiple magnets may be configured such that the forces exerted on the primary magnet(s) that move with the FSD system 200 are directly proportional, or as close as possible to directly proportional, to the displacement of the FSD system 200. In some embodiments, a magnetic field created by the one or multiple magnets may be configured such that the non-linearity of the forces exerted on the primary magnet(s) that move with the FSD system 200 are reduced or minimized.

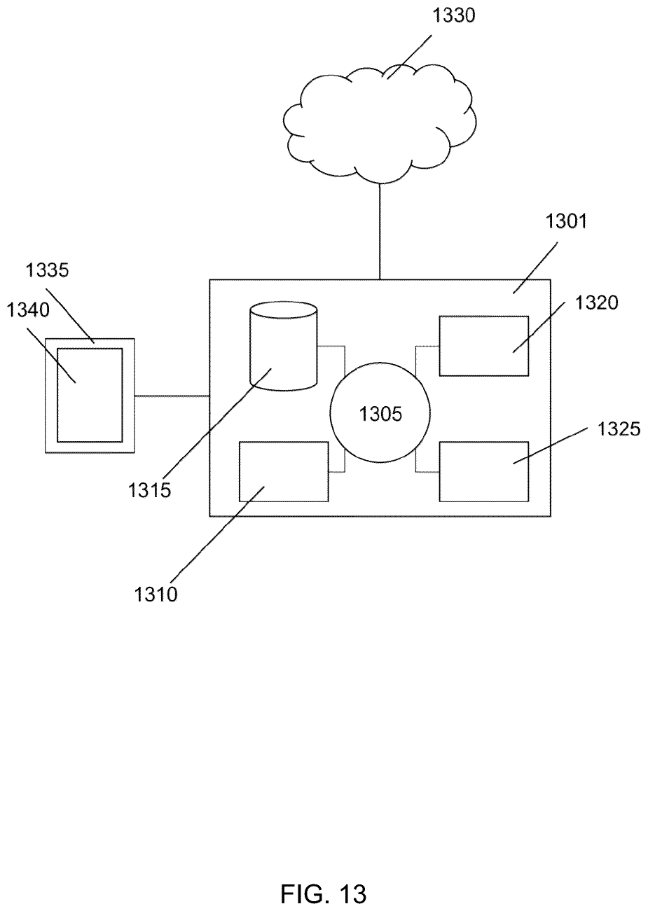

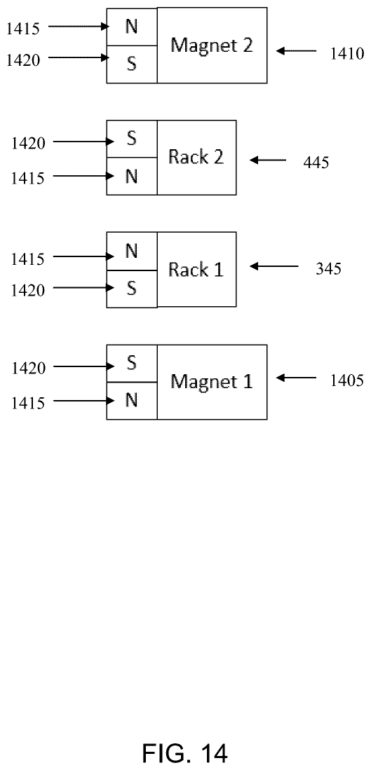

In some embodiments, the one or multiple magnets may be configured to at least partly replicate or approximate the spring-like behavior of linear springs obeying Hooke's law. Referring to FIG. 14, bar, cylinder, or other shaped magnets may be placed on or comprise the rack 345 in the first rack and pinion system 340. Bar, cylinder, or other shaped magnets may also be placed on or comprise the rack 445 in the second rack and pinion system 440. The magnets may be oriented such that the north poles 1415 on the two racks face one another to produce a repelling force between the racks. In some embodiments, the south poles 1420 on the two racks may face one another. A magnet 1405, 1410 may be placed on the wearable base 110 or other component on the opposite side of each rack from the other rack, such that the pole facing the rack is the same as the pole on rack facing the magnet. For instance, referring the FIG. 14, the south pole 1420 of Magnet 2 1410 on the wearable base 110 or other component may face the south pole 1420 of the magnet on rack 2 445. The south pole 1420 of Magnet 1 1405 on the wearable base 110 or other component may face the south pole 1420 of the magnet on rack 1 345. This may generate a repelling force between the magnets on the rack and the magnets on the wearable base 110 or other component. As the two racks move toward one another, the repelling force between the two racks may increase. This dynamic may be similar to the case in which a compression spring exists between the two racks. As the racks move farther apart from each other, the repelling force between the two racks may decrease, but the repelling force between Magnet 2 1410 and Rack 2 445 and the repelling force between Magnet 1 1405 and Rack 1 335 may increase. The net forces on each rack may be in the direction causing the racks to move towards one another. This dynamic may be similar to the case in which an extension spring exists between the two racks. In this way, magnets may replicate or approximate spring-like behavior. In some embodiments, similar magnet configurations may be placed on other force transmission systems 120.

In some embodiments, electromagnet(s) polarity may be changed by changing the direction of the current, for instance, which may determine the direction of force acting on it. For instance, at least one permanent magnet may be fixedly attached to a first rack and pinion system 340 (part of a force transfer system 120 described in later section) that is located between the hand H and the spring system 220, such that the permanent magnet(s) move with the first rack and pinion system 340 as forces are transmitted between the hand H and spring system 220. At least one electromagnet may be fixedly attached to a second rack and pinion system 440 that is meshed with the pinion 245 in the flywheel-mechanical advantage system 400, such that the electromagnet(s) move with the second rack and pinion system 440 as forces are transmitted to the flywheel system 210 and spring system 220. The electromagnet(s) may interact with the magnetic fields generated by the presence of the magnet(s) and exert a force on the rack and pinion system 440 it is attached to. The direction of force may depend on the magnet(s) polarity at the time. Depending on the cycle during the out-of-phase motion of the flywheel system 210 and hand H (e.g., whether the hand H is rotating upwards or downwards, or the flywheel 210 is rotating clockwise or counterclockwise), the polarity of the electromagnet may be altered to generate attractive or repulsive forces between itself and the other magnets. In this way, the system may act as a spring in the FSD system 200. One or multiple of these configurations may be used to configure the system such that the effective spring constant is linear (in that the force-displacement relationship is linear) or non-linear to a specified degree or range. For instance, the electromagnets may be fixed in place on the wearable base 110 and the permanent magnets on other moving components in the device 100. In some embodiments, two electromagnet(s) may be fixedly attached to the two racks in the rack and pinion systems.

In some embodiments, one or multiple forms of coils may surround the magnetic spring systems. The multiple forms of coils may including but are not limited to Solenoid Coils, Induction Coils, Toroidal Coils, Air-Core Coils, Iron-Core Coils and/or Pancake Coils. In some embodiments, the coil(s) may be part of an electrical circuit that includes the magnetic springs. In some embodiments, no electric current may be applied externally through the surrounding coils. The movement of the magnets through the surrounding coil may generate eddy currents in the coil, which may generate their own magnetic fields and thus affect the system's current magnetic field. This may affect the strength and direction of forces exerted on the magnets and thereby affect the resulting motion of the flywheel and hand tremor. The presence of the coil may also cause magnetic distortion or magnetic friction. The movement of the magnets may induce a voltage across the coil and this energy may be used in an external system to impact the movement of and force exerted on the flywheel and hand tremor systems. This is described in a further section.

In some embodiments, an electric current may be applied externally through the coil temporarily or permanently. When applied, it may alter the behavior the magnetic spring system by affecting the strength and direction of the forces on the magnets, thereby affecting the resulting motion of the flywheel system 210 and hand H. It may also be used to calibrate and provide temporary control or manipulation forces to fine-tune the system. This is described further in the next section.

### Frequency Adjustment Systems

Characteristics of a user's tremor motion (i.e., frequency and/or amplitude) may change during and/or between tremor episodes. In some embodiments, a device 100 as described herein, may comprise sensors and a control unit configured to sense, record, and analyze data related of the characteristics and changes in the characteristics in a user's tremor motion. In some embodiments, the sensors may include but are not limited to accelerometers, gyroscopes, motions sensors, and other sensors. In some embodiments, the sensors and control unit may be configured to autonomously, or with the aid of external devices, calculate a new tremor frequency and amplitude. In some embodiments, the control unit may be configured to analyze the sensed data and determine in what way and/or to what extent the configuration of the FSD system(s) 200 should be adjusted to most effectively treat the user and reduce the amplitude of the new tremor pattern. The control unit may be configured to execute instructions to control the device 100 and adjust the FSD system(s) 200 in response to determining a change or configuration to optimize treatment and tremor dampening. Not adjusting to a tremor frequency change may result in the FSD system 200 operating sub-optimally and may reduce its efficacy in reducing the amplitude of the hand tremor. The control unit may be configured to adjust the FSD system 200 due to a tremor frequency change. The adjustment may allow the FSD system 200 to maintain or increase its efficacy in reducing the amplitude of the hand tremor. In some embodiments, the control is configured to adjust one or multiple settings and/or configurations (i.e., friction, tension, spring constant, effective mass, etc.) of the device as described herein. In some embodiments, the control unit is configured to automatically adjust one or multiple settings of the device in response to a sensed signal. The one or more adjustments may be to optimize the device to treat a user's tremor. In some embodiments, the control unit is configured to adjust one or multiple settings in response to a user instruction. In some embodiments the control unit may be an external device. In some embodiments, the control unit may be configured to receive and execute one or more programable instructions.

A method that may be used to adjust the FSD system 200 in response to a change in tremor frequency comprises adjusting an effective mass of the flywheel 210. In some embodiments, the effective mass of the flywheel 210 may be configured to be controlled by manipulating or adjusting a mechanical advantage mechanism 400 that acts on the flywheel 210. In some embodiments, increasing the extent of the mechanical advantage 400 on the flywheel 210 may cause the flywheel's 210 angular displacement to increase or be amplified for a given input displacement of a rack 445 in a second rack and pinion system 440, which may increase the flywheel 210 effective mass. In some embodiments, decreasing the extent of the mechanical advantage on the flywheel 210 may decrease the flywheel 210 effective mass.

In some embodiments, increasing the flywheel 210 effective mass may cause the FSD system 200 to be more effective at counteracting a lower frequency tremor. In some embodiments, decreasing the flywheel 210 effective mass may cause the FSD system 200 to be more effective at counteracting higher frequency tremor.

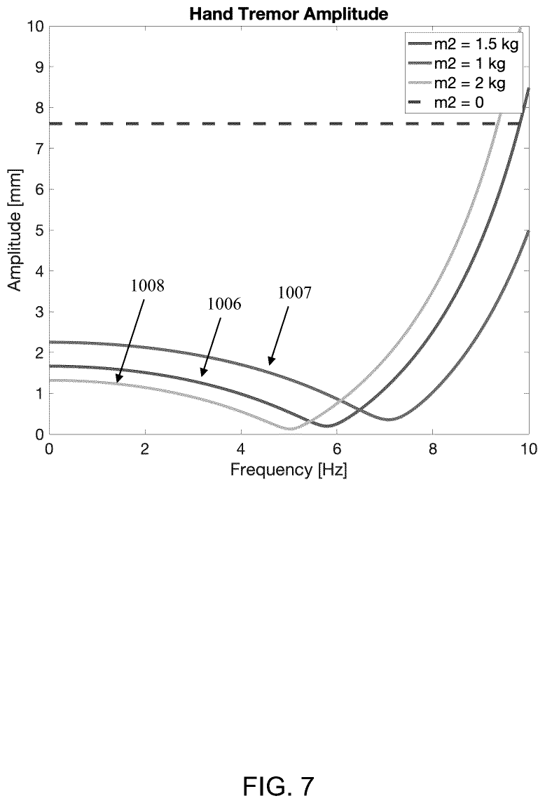

Referring to FIG. 7, an FSD system 200 comprising a flywheel 210 having an effective mass of 1.5 kg may be optimized to reduce tremor amplitude for a subject experiencing tremors of frequency about 5.8 Hz, as shown by curve “m2=1.5” 1006. If the tremor frequency increases to 7.1 Hz, the flywheel 210 effective mass may be decreased to 1 kg to best counteract the new tremor frequency, all else kept equal, as shown by curve “m2=1” 1007. If the tremor frequency decreases to 5.0 Hz, the flywheel 210 effective mass may be increased to 2 kg to best counteract the new tremor frequency, all else kept equal, as shown by curve “m2=2” 1008.

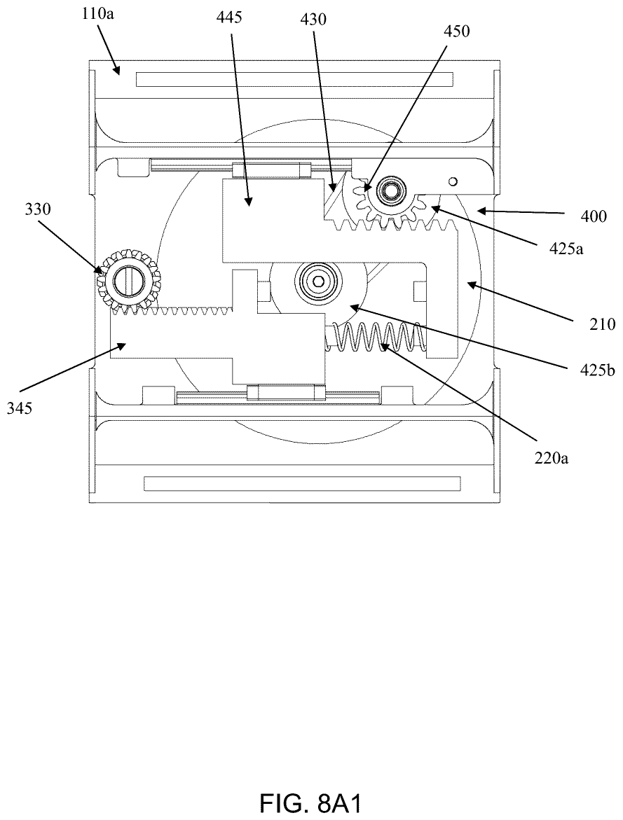

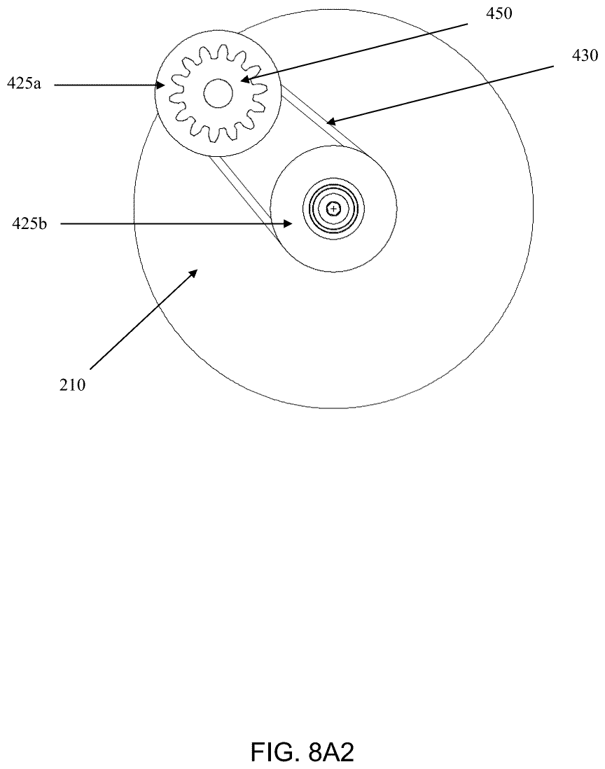

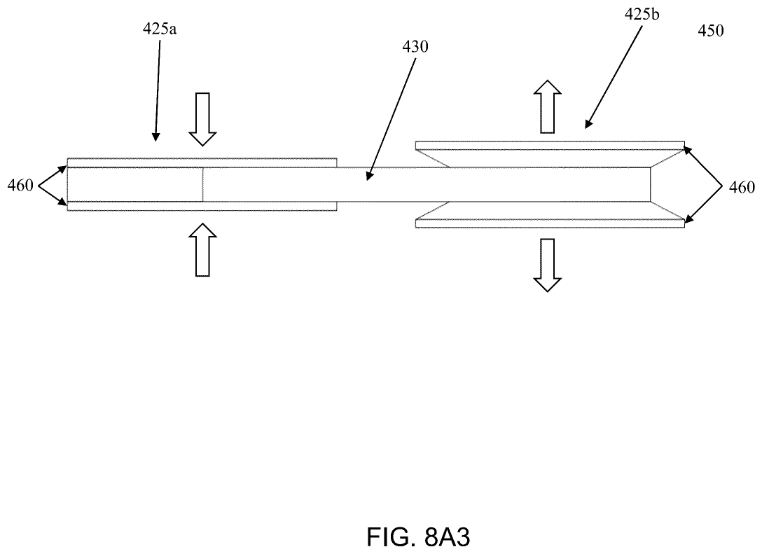

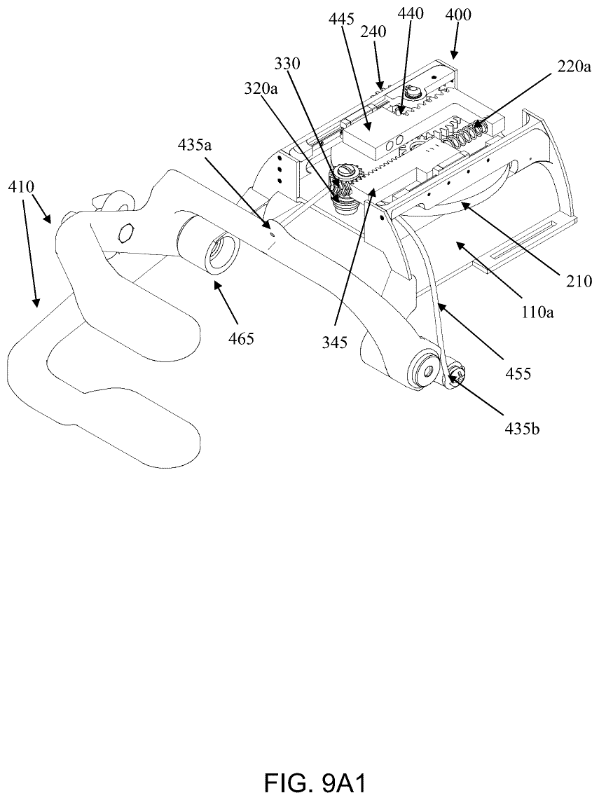

In some embodiments, the device 100 may comprise one or multiple adjustment mechanisms configured to adjust the extent of a mechanical advantage system 400 acting on the flywheel 210. The one or multiple adjustment mechanisms may include a continuously variable transmission (CVT) system. The CVT system may be configured to change a gear ratio applied on the flywheel 210. FIGS. 8A1-8A3 illustrates non-limiting examples of the FSD 200 comprising a CVT system as described herein. In some embodiments, the CVT system may comprise two adjustable pulleys (i.e., a first pulley and a second pulley) with a belt 430 (e.g., V-belt) configured for transmitting the motion between the two adjustable pulleys. In some embodiments, the adjustable pulleys may be configured to adjust the distance between the sheaves of each pulley. The first pulley may be a CVT driving pulley 425a and the second pulley may be a CVT driven pulley 425b. The driving pulley 425a may be attached to, or be part of, a force transmission system (such as the force transmission system 120 illustrated in FIG. 1) from the hand H to the flywheel 210. The driven pulley 425b may be coupled to the flywheel 210. In some embodiments the driven pulley 425b may be rigidly coupled to the flywheel 210. In some embodiments, the driven pulley 425b may be directly or indirectly coupled to the flywheel 210.

In some embodiments, the mechanical advantage delivered to the flywheel 210 may be affected by the gear ratio of the CVT system. In some embodiments, the CVT system may be configured to control the gear ratio by changing a distance between two sheaves of the pulleys, such as the driving pulley 425a and the driven pulley 425b. In some embodiments, the CVT system is configured to increase and/or decrease the distance between the sheaves of each pulley. In a non-limiting example, when a distance between the sheaves of one pulley increases the distance between the sheaves of the other pulley may be decreased.

Referring to FIGS. 8A1-8A3, in some embodiments, the driving pulley 425a may be connected to the second rack and pinion system 440 via a pinion 450 that is rigidly attached to the driving pulley 425a. The driven pulley 425b may be rigidly attached to the flywheel 210.

Referring to the non-limiting example illustrated in FIGS. 8A2-8A3, the distance between the sheaves 460 of the driving pulley 425a may be decreased, causing the length over which the belt 430 is in contact with the driving pulley 425a to be increased. The distance between the sheaves 460 of the driven pulley 425b may be increased, causing the length over which the belt 430 is in contact with the driven pulley 425b to be decreased. This configuration may be thought of as increasing the effective diameter of the driving pulley 425a and decreasing the effective diameter of the driven pulley 425b. This may change the gear ratio, or drive ratio, of the CVT system such that it may increase the output speed relative to the input speed. This may increase the effective mass of the flywheel 210. One or multiple idler pulleys may be used in the CVT setup.

In another embodiment, the distance between the sheaves 460 of the driving pulley 425a may be increased, causing the length over which the belt 430 is in contact with the driving pulley 425a to be decreased. The distance between the sheaves 460 of the driven pulley 425b may be decreased, causing the length over which the belt 430 is in contact with the driven pulley 425b to be increased. This configuration may be thought of as decreasing the effective diameter of the driving pulley 425a and increasing the diameter of the driven pulley 425b. This may change the gear ratio, or drive ratio, of the CVT system such that it may decrease the output speed relative to the input speed. This may decrease the effective mass of the flywheel 210.

Variations of the CVT systems described above may be employed. In a non-limiting example, a chain-based design may be used place of or in combination with a belt-driven design. In some embodiments, a ratcheting, toroidal, cone, epicyclic or hydraulic CVTs may be used.

In some embodiments, a device or system 100 as describe herein, may further comprise one or multiple pulley adjustment mechanisms configured to adjust a distance between the sheaves 460 of the pulleys 425. In some embodiments, the pulley adjustment mechanisms may be configured to automatically, semi-automatically or manually adjust the distance between sheaves 460. The pulley adjustment mechanisms may comprise one or multiple mechanical linkages. The one or multiple linkages may comprise levers or rods attached to the pulleys configured to be moved for increasing the distance between the sheaves 460 in one pulley and decreasing the distance between the sheaves and the other pulley. In some embodiments, the tension of the belt 430 may be configured to be adjusted to adjust the sheave distance. In some embodiments, the tension of the belt 430 may be adjusted using at least one of a tensioner pulley, an actuator, or spring-loaded mechanism. Adjusting the tension of the belt 430 may be configured to change a rest position relative to the driving pulley 425a and/or driven pulley 425b. This may be used to change the gear ratio of the CVT. In some embodiments, a threaded screw mechanism, as described in the spring coil restrictor section, may also be used to control sheave distance. In some embodiments, the threaded screw may be configured to increase the distance between the sheaves when rotated in a first direction and decrease the distance between the sheaves when rotated in a second direction opposite the first direction. In some embodiments, a ball-screw, lead-screw or roller-screw mechanism may enable the threaded screw mechanism. In some embodiments, one or multiple motors and/or actuators may be used to control and adjust the distance between the sheaves 460 of the two pulleys. In some embodiments, one or multiple ratchet and pawl systems may be configured to be coupled to the sheave(s) via cable(s) to change and hold different positions. In some embodiments, the tilt angle between the discs in a toroidal CVT may be adjusted to control the drive ratio. In some embodiments, pneumatic actuators may be used to control sheave distance or disc angle. In some embodiments, the user may manually change the position of the sheaves.

### Planetary Gear System

In some embodiments, a planetary gear system may be used to change the extent of the mechanical advantage system 400 acting on the flywheel 210. The planetary gear system may comprise a sun gear, one or more planetary gears, one or more ring gears, and one or more carriers. In some embodiments, the carrier may be configured to be connected directly or indirectly to the flywheel 210. In some embodiments, the sun gear may be configured to be connected directly or indirectly to one or more force transmission(s) systems. In some embodiments, adjusting the speed of the ring gear, may be used to control the output speed of the planetary gears and carrier. In some embodiments, increasing the speed of the ring gear when it is rotating in the same direction as the sun gear, or decreasing the speed of the ring gear when it is rotating in the opposite direction as the sun gear, may increase the carrier output speed. In some embodiments, decreasing the speed of the ring gear when it is rotating in the same direction as the sun gear, or increasing the speed of the ring gear when it is rotating in the opposite direction as the sun gear, may decrease the carrier output speed. In accordance with the non-limiting examples described above, the gear ratio of the planetary gear system, and thus flywheel 210 effective mass, may be controlled. In both non-limiting examples above, it is assumed that the ring gear is not rotating such that the carrier is rotating in the opposite direction as the sun gear.

In some embodiments, a carrier output speed may be adjusted and/or controlled. Increasing the carrier output speed may amplify the motion of the flywheel 210, thereby increasing its effective mass during a tremor. Decreasing the carrier output speed may decrease the amplification of the flywheel 210 motion, thereby decreasing its effective mass during a tremor.

In some embodiments, one or multiple actuators and/or motors may be used to change the speed of a ring gear. The ring gear may also be coupled to other rotating and/or stationary components of a wearable base of the device 100. In some embodiments, a CVT, as described herein, may be attached to the ring gear and configured to control the rotational speed of the ring gear. In some embodiments, one or multiple brake or clutch mechanisms may be used to control or prevent ring gear motion. In some embodiments the one or multiple actuators or motors may be configured to engage and disengage the ring gear from said rotating and/or stationary components. In a non-limiting example, engaging the ring gear with a stationary component may keep the ring gear stationary. In some embodiments, multiple intermediary planetary gear systems may be configured to transmit a mechanical advantage to the flywheel 210. In some embodiments, engaging and/or disengaging one or multiple ring gears, and/or controlling their speeds, may be used to adjust the effective mass of the flywheel 210. In some embodiments, the user may manually couple the ring gear to another component or otherwise affect the speed of the ring gear.

### Chain/Belt Drives

In some embodiments, a mechanical advantage system may comprise belt drive systems or chain drive systems. The belt drive systems may comprise a belt configured to be wrapped around pulleys. The chain drive systems may comprise a chain configured to be wrapped around sprockets. A gear ratio of the belt drive system may be controlled by changing the diameter of the pulleys in the belt drive system. A gear ratio of the chain drive system may be controlled by changing the diameter of the sprockets in the chain drive system. In some embodiments, the belt drive system may comprise step pulleys configured to move a belt from one pulley diameter to another. In some embodiments, spring loaded tensioners, or other automatic or semi-automatic tensioning mechanisms may be configured to maintain belt tension in the belt driver system. In some embodiments, manual tensioning mechanisms configured to maintain belt tension in the belt driver system. In some embodiments, cone pulleys may be used to change gear ratios in belt drives. The tensions of chain and sprockets of the chain driver system may be adjusted using the same or similar techniques as described above for the belt driver system. In some embodiments, the user may manually change the position of the belt from one pulley to another.

### Gear Shifting Mechanisms

In some embodiments, an apparatus, device, or system as described herein may comprise a gear-shifting mechanism. The gear shifting mechanism may comprise a derailleur system and/or an internal gear hub system, similar to in a bicycle. In some embodiments, the gear-shifting mechanism may be used to change a gear ratio and the extent of a mechanical advantage mechanism 400 acting on the flywheel 210. In some embodiments, an internal gear hub system may be configured to alter the tension of shifting cables in a shifting mechanism to change gears. In some embodiments, pulling or releasing of tension in the shifting cables may be configured to move pawls that are engaged with gear(s) in a planetary gear system such that they engage with different gear(s) in the system. The different gear engaged may be of a different diameter, which may change the gear ratio in the mechanical advantage mechanism applied to the flywheel 210, which may in turn affect the flywheel 210 effective mass. In some embodiments, the user may manually select or change the engaged gear in the system.

In some embodiments, the gear-shifting mechanism may be configured so that the user may manually change the gears and/or gear ratios in the system. The user may adjust the gears and/or gear ratios to calibrate the device to their tremor during or between tremor episodes. In some embodiments, the device 100 may also automatically initiate a gear or gear ratio change to optimize the FSD system 200 configuration to best counteract the tremor.

A method that may be used to adjust the FSD system 200 in response to a change in tremor frequency comprises adjusting an effective spring constant of the spring system 220. In some embodiments, the device 100 may be configured to automatically, semi-automatically, or manually adjust and/or reconfigure one or multiple FSD systems 200 in response to a tremor frequency change. The spring system 220 may be manipulated such that the effective spring constant is increased or decreased. The device 100 may be configured to make the spring elements(s) (i.e., 220a, 220b, or 220c) of the spring system 220 more “linear” or more “non-linear”. In a non-limiting example, if a frequency of a user's tremor decreases, the effective spring constant of the spring system 220 may in response be decreased, whereas if the frequency of the tremor increases, the effective spring constant of the spring system 220 may be increased. By changing the effective spring constant of the spring system 220, the out-of-phase harmonic motion of the flywheel 210 may be altered such that the new out-of-phase motion better interferes with the new hand tremor motion.

In some embodiments, one or multiple spring adjustment mechanisms may be configured to adjust the effective spring constant of the spring system 220. The one or multiple spring adjustment mechanisms may be used in isolation or in combination with one another to achieve a new effective spring constant of the spring system 220. The mechanism used may depend on the type of spring system 220. In spring systems 220 involving traditional coil springs, one or multiple mechanisms that change the physical or active properties of the spring elements, or adding components to the spring elements, may be employed.

The number of active coils in the spring elements 220 may be altered. Active coils in the springs are the coils that are able to deform and/or move during spring deformation, and may contribute to the storing and release of the spring's potential energy. The number of active coils in one or multiple spring elements may be decreased to increase the effective spring constant of the spring system 220. Similarly, the number of active coils in one or multiple spring elements may be increased to decrease the effective spring constant of the spring system 220. One or multiple motors and/or actuators may be used, for instance, to restrict the number of active coils in the spring system 220. Similarly, the one or multiple motors and/or actuators may be used to release certain coil and allow for a greater number of active coils in the spring system 220. For instance, actuator(s) may be connected to cables, which may pass through one or multiple locations in the coils in the spring(s). The movement of actuator(s) in one direction may restrict the coils from moving within the system, while the movement of actuator(s) in the other direction may allow the coils to move freely in the system. In some embodiments, the user may manually change the position of the actuators to affect cable tension. In some embodiments, one or multiple locking mechanisms may be employed, where a lever, motor, or actuator may restrict or release the spring coils. One or multiple clamps or belts may be used, such that when closed or tightened by an actuator, they may restrict the coils, and when opened, they may release the coils.

In some embodiments, the one or multiple spring adjustment mechanisms comprise one or multiple adjustable collar or sleeves mechanisms. The one or multiple adjustable collar mechanisms may be configured to control and/or adjust an overall spring length and/or restrict active coils, of one or more spring elements of the spring system 220 and thus change the effective spring constant of the spring system 220. The adjustable collar(s) or sleeve(s) mechanisms may be wrapped around a one or more number of coils in one or multiple springs. The adjustable collar(s) or sleeve(s) mechanisms may also be configured to slide along length of the springs of the spring system 220. The collar(s) may comprise a locking mechanism. The locking mechanism may include but is not limited to set screws, clamps and/or threaded nuts and bolts. Tightening the collar may compress the spring and reduce its effective length and/or restrict the extent to which the active coils may deform. This may increase the effective spring constant. Similarly, loosening or decreasing the extent to which the collar is compressing the spring and/or restricting coil movement or deformation may decrease the effective spring constant.

In some embodiments, the one or multiple spring adjustment mechanisms comprise one or multiple threaded rod mechanisms. The one or multiple threaded rods may be configured to run along the length of the spring(s). Rotating the threaded rod(s) in a first direction may compress at least one section of the spring. This may decrease the effective length and/or the number of coils that become inactive or less engaged during spring deformation, which may increase the effective spring constant. Rotating the rod in the other direction may decompress or even extend the at least one section of the spring, and may decrease the effective spring constant.

In some embodiments, the one or multiple spring adjustment mechanisms comprise a lever system. The lever system may comprise one or multiple lever arms that may be configured to rotate about their respective pivot points and are connected to spring element(s) at one or multiple locations. In some embodiments, one or multiple motors or actuators may be configured to exert forces on the lever(s), to compress and/or expand sections of the spring or the entire spring itself. This may restrict or release active coils in the spring to change the effective spring constant. In some embodiments, the user may manually configure the lever system to the desired position.

In some embodiments, the one or multiple spring adjustment mechanisms may be configured to engage and/or disengage one or multiple springs of the spring system 220. The spring adjustment mechanisms may control the effective spring constant of the spring system 220, based on which springs are engaged or disengaged and/or whether the engaged/disengaged springs are in parallel or in series with the other spring elements. In a non-limiting example, disengaging a spring element that is in parallel with the other spring elements may decrease the effective spring constant of the spring system 220. In some embodiments, the user may manually change engage or disengage springs. In some embodiments, the one or multiple spring adjustment mechanisms may be configured adjust and/or control the angle of action of the spring elements of the spring system 220. The angle of action may be the angle between the axis of the spring and the axis of motion of the relevant force transmission systems. In some embodiments, adjusting the angle of action of the spring elements may be used to alter or affect the effective spring constant of the spring system 220. In a non-limiting example, the angle of the spring(s) 220a and/or 220b between the first rack and pinion system 340 and second rack and pinion systems 440 may be altered such that the spring(s) 220a and/or 220b is no longer parallel to the normal plane of the spring system 220. The degree to which this angle deviates from being parallel to the plane may impact the degree to which the spring constant is changed. In some embodiments, the user may manually change the angle of action. In some embodiments, vertical or horizontal sliders connected via a connecting rods or other force transmissions mechanisms may move along springs and restrict coil movement.

### Threaded Cylinder Mechanism

In some embodiments, the spring adjustment mechanism may comprise continuous spring barriers. The continuous spring barriers may comprise barriers or separators configured to create a separation between active and inactive spring coils. In some embodiments, the continuous spring barriers include but are not limited to threaded collars, plates, or containers configured to partition the spring into at least a first set and second set of spring coils. In some embodiments a first set of spring coils may be inactivated, and a second set of spring coils may be active. In some embodiments, the first set of inactive spring coils may be configured to not deflect, or partially deflect, in response to forces acting on the spring. The second set of active spring coils may be configured to deflect normally, unrestricted, or minimally restricted, in response to forces acting on the spring. In some embodiments, the spring adjustment mechanism is configured to control and/or adjust a ratio of the number of inactive spring coils to the number of active spring coils. Adjusting the ratio of inactive spring coils and active spring coils may be used to control and/or adjust a spring constant of the spring element(s).

In a non-limiting example, a plate with at least two holes may comprise a barrier. A spring coil may pass through the first hole at a given position such that the plate may be configured to separate the spring into a first and second sections. The second hole may be configured to act as an attachment point for a motor shaft, actuator, or other mechanism such as a ball screw or roller screw mechanism. In some embodiments, a traditional or helical ball screw mechanism may be used in which the movable nut may be rigidly attached to the plate via the second hole. The second hole may further include a stopper configured to hold the spring in a position. The stopper may comprise a rubber-like material. Variables such as hole locations, spring diameter, and motor diameter may be configured such that, as the plate translates linearly, it also rotates in a way that the first hole follows the path along the coils of the spring. The plate may be configured to change its position along the length of the spring. Changing the position of the plate along the spring may affect the ratio of active to inactive coils in the spring. In some embodiments, the first set of spring coils proximal to a first rack and pinion system may be inactive, and the second set of spring coils distal to the first rack and pinion system may be active. The first set of spring coils may be prevented from fully deflecting. In some embodiments the first set of spring coils may not be able to deflect at all. If the plate is repositioned such that there is a greater number of inactive coils than in the position before, the spring constant may be increased. Likewise, if the plate is repositioned such that there is a lower number of inactive coils than in the position before, the spring constant may be decreased. In some embodiments, the user may manually change the position of the barrier along the spring.

In some embodiments, at least part of the spring may be confined in a container such that the coils positioned in the container are inactive. In some embodiments, the container may comprise a hollow, externally threaded cylinder or tube. A threaded collar may be affixed to the wearable base or other component such as the rack 345 in the first rack and pinion system 340. The threaded tube may have cap(s) on at least one end with at least one hole in the cap(s). The external threads of the threaded tube may engage with the internal threads of the threaded collar, such that at least part of the threaded tube is located within at least part of the threaded collar. The spring may pass through the hole in the threaded tube. The threaded tube may translate linearly as it rotates along the threaded collar. The hole in the cap of the threaded tube may be positioned such that as the threaded tube rotates and linearly translates away from the threaded collar, it may enclose more spring coils as the hole travels along the path of the spring. As the threaded tube rotates and linearly translates toward the threaded collar, it may enclose fewer spring coils. The coils contained in the threaded tube may be inactive and be prevented from normally or fully deflecting in response to forces acting on the spring. Helical gear lead, ball, or roller screw mechanisms, motors, and actuators for instance, may enable the mechanics of this spring coil restrictor system. In some embodiments, the user may manually change the position of the threaded tube.

### Gear Spring Restrictor

Other mechanisms that may be used to restrict and release active coils in the spring may employ one or multiple gears and/or pawls. For instance, a gear may be attached to the wearable base or other components such as the rack 345 in the first rack and pinion system 340. The gear may be positioned such that one or multiple gear teeth extend at least partly in the area in between consecutive coils of the spring. The spring coils on one side of the gear may be restricted from deflecting, while coils on the other side of the gear may be free to deflect. The number of coils restricted from deflecting may impact the spring constant of the system. The rotation of the gear in one direction may pull a spring coil away from the length of the spring and hold it such that it is prevented from deflecting. The next gear tooth may then be positioned such that it extends at least partly in the area between the next two consecutive spring coils. In this way, rotating the gear in this direction may restrict coils from deflection. The rotation of the gear in the other direction may release coils that are already restricted. In some embodiments, the user may manually change rotate the gear such that the desired coils are restricted from deflecting.

### Ratchet Pawl Mechanism

In some embodiments, a ratchet and pawl mechanism may also be used to restrict active spring coils and manipulate the spring system's effective spring constant. For instance, a ratchet wheel may be mounted on a shaft attached to the wearable base or other component such as a motor. A cable may be attached to the ratchet wheel or other component such as a gear that is also mounted on the shaft. The cable may be wrapped around one or multiple sections of the spring such that when the cable is tightened, the coils in those sections of the springs may be prevented from deflecting. A spring-loaded pawl may have at least one tooth engaged with the rachet wheel. When the ratchet wheel and/or gear is rotated in one direction, the cable may be tightened. Once the cable is tightened to the desired tension, the ratchet wheel and/or gear may stop rotating and the pawl may engage with the ratchet wheel to prevent the gear from rotating in the opposite direction. The pawl may be disengaged to allow the gear to rotate in the opposite direction and loosen the cable, allowing the coils in those sections of the spring to deflect to at least some degree. The extent to which the cable is tightened may affect the extent to which the spring coils may be able to deflect. The greater extent to which the cable is tightened, the lesser degree to which those coils may be able to deflect. Similarly, the lesser degree to which the cable is tightened, the greater degree to which those coils may be able to deflect. In some embodiments, the user may manually rotate the ratchet wheel to the desired position to affect cable tension. In some embodiments, the user may manually change the spring coils over which the cable is engaged with or wrapped around.

One or multiple mechanisms may be used to disengage the pawl. The force of the spring or inertia of the pawl may be overcome to disengage the pawl. For instance, a secondary actuator or motor acting directly or indirectly on the pawl may overcome this spring force. The rotation of this secondary motor may, for instance, pull on a cable attached to the pawl to disengage it from the ratchet wheel. A CAM-driven release mechanism may also be used to disengage the pawl. For instance, a CAM or CAM-follower mechanism may disengage the pawl from the ratchet system when rotated or moved to certain positions. Another mechanism that may be used is a magnetic release, where a magnet may hold the pawl such that it is engaged with the ratchet wheel. A change in the surrounding magnetic field may disengage the pawl from the ratchet wheel. An electromechanical solenoid actuator may also be used to disengage the pawl from the ratchet wheel by pushing the pawl tooth away from the ratchet wheel. In some embodiments, the user may manually disengage the pawl tooth.

### Cable-Enabled Spring

One or multiple spring elements may also be attached to at least one force transmission systems via cable joints. For instance, a spring may be fixedly attached to the wearable base on one side such that its axis is perpendicular or at an angle to the axis of motion of the rack in the first rack and pinion system. On the other side, the spring may be attached to one end of the rack in the first rack and pinion system via a cable in tension. One or multiple pulleys or other components may facilitate and/or guide the cable path between the spring and rack. At rest, the spring may be positioned in the middle of the rest length of the spring. The cable may be inelastic such that increasing the cable tension does not increase the cable length or does so minimally. The motion of the rack in the forward direction, for instance, may pull on the cable, which may then pull on and extend the spring. As a result of the spring extension, the spring may exert a force on the cable in the other direction. This force may be felt by the rack which may resist the motion of the rack in the forward direction. The greater the spring is extended, the greater the resisting force. Similarly, the force exerted by the spring on the rack via the cable may resist the motion of the rack if it moves in the backward direction. A pre-tension in the cable may create a pre-load in the spring. One or multiple springs may be configured in this manner with respect to the force transmission systems. These springs may have coil restrictor mechanisms acting on it to change the spring constant. The springs may be disengaged by disengaging the cable connecting the spring to the rack, for instance. In some embodiments, the user may manually change the cable tension.

In some embodiments, cables or straps may be wrapped around certain section(s) of the spring. The cables or straps may be attached on at least one end to a motor or actuator. The rotation of a motor in one direction or the displacement of an actuator in one direction, may tighten the cables or straps such that they prevent coil deflection in that section of the spring. The rotation of a motor in the other direction or the displacement of an actuator in the other direction, may loosen the cables or straps such that they allow coil deflection.

### Spring Overlay Rack

In some embodiments, one or multiple springs elements may overlay one or more components of the force transmission system. For instance, at least part of a spring may encompass the rack 345 in the first rack and pinion system 340. Similarly, at least part of the spring may be inside the rack 345 in the first rack and pinion system 340. In some embodiments, the spring axis may be parallel to the axis of motion of the first rack and pinion system 340. For instance, the spring may be positioned directly above, below or to the sides of the rack 345. This may reduce the overall space taken up by the components in this direction.

In some embodiments, combinations of one or multiple of spring systems 220, as described herein may be employed. The multiple spring systems 220 may be used in parallel and/or in series. In some embodiments, a specific combination or configuration of the one or multiple spring systems 220 may be configured to control one or more of an effective spring constant, linear or non-linear, in the system at particular positions and/or times.

### Alternative Spring System Frequency Adjustment Mechanisms

In the case of gas springs or alternative gas spring systems, as described herein, the gas pressure in the cylinders or tubing may be altered. In a non-limiting example, increasing the gas pressure may increase the effective spring constant, while decreasing the gas pressure may decrease the effective spring constant. One or multiple diaphragm pumps, pressure sensors, gas supply systems and/or pressure relief valves may be used to control the gas pressure. Increasing gas supply in the tubing, increasing the gas temperature, and/or externally compressing the tubing (e.g., laterally) may increase the gas pressure.

In the case of antagonistic spring systems, pre-loads may be applied to the system such that the equilibrium position and/or the balance of forces between the opposing springs, may be affected. This may impact the effective spring constant of the system. Increasing the magnitude of the pre-load may increase the effective spring constant, while decreasing the magnitude of the pre-load may decrease the effective spring constant; this may not always be the case, however, and may depend upon the specific configuration of the antagonistic spring system. Additionally, the methods of changing the effective spring constant of coil springs described earlier may be applied to at least one spring in the antagonistic spring system. In some embodiments, the user may manually change the pre-load.

In the case of hydraulic systems, as described herein, the effective fluid bulk modulus may be adjusted. In some embodiments, the effective fluid bulk modulus of electrorheological or magnetorheological fluids may be changed by applying or changing an electrical or magnetic field, respectively. In some embodiments, the fluid bulk modulus may be changed by using multiple fluids in separate hydraulic springs systems and controlling which combination of systems are used. The geometry of the hydraulic system may be adjusted to change the effective spring constant. Adjusting the effective cross-sectional area of the hydraulic cylinder may change the effective spring constant. In some embodiments, increasing the effective cross-sectional area may increase the effective spring constant. In non-limiting examples, variable cross section area pistons or embedded pistons may be used and/or controlled to change effective cross-sectional area. In some embodiments, fluid may be added to the system to increase fluid volume. Increasing fluid volume may decrease effective spring constant. In some embodiments, hydraulic fluid pressure may be adjusted to change the effective spring constant. In some embodiments, the effective spring constant may be directly proportional to the hydraulic fluid pressure. In a non-limiting example, a hydraulic pump may be used to adjust fluid pressure.

In the case of magnetic spring systems, constant or time-varying electric currents may be passed in the coils surrounding or interacting with the magnetic spring system, or in one or more electromagnets in the system. The flow of the electric current may generate a magnetic field around the coil, which may interact with the existing magnetic field that affects the behavior of the magnetic spring system. The new magnetic field may increase or decrease the effective spring constant of the system. This may depend on the existing magnetic field orientation, distribution and strength, the direction and strength of the electric current, and the orientation, distribution, and strength of the newly generated magnetic field. By controlling these parameters, the effective spring constant of the system may be altered. In some embodiments, a magnetic field applied or changed in the presence of magnetorheological or ferrofluids fluids may change the effective spring constant delivered by the fluid, in a hydraulic spring system, for instance.

In some embodiments, the movement of magnets through the one or multiple coils in the system may induce a voltage across the coil(s). In some embodiments, the coil or conductor may be moving relative to the magnets. The induced voltage may be used to generate an electric current that may be used for various purposes in the device. For instance, it may be used for energy harvesting and may power one or multiple actuators in mechanisms that may change the system effective spring constant, as described earlier. It may also power one or multiple sensors, processors, and/or other electrical components in the device, as described later.

One or multiple of adjustment systems (i.e., spring adjustment systems) as described herein may be used in insolation or in combination with one another.

### Force Transfer Systems

An apparatus, device, system or method as described herein may comprise one or multiple force transmission system(s) 120. The one or multiple force transmission system(s) 120 (also referred to as force transfer system(s) 120 herein) may be configured to transmit the force and motion between a first and second elements and/or components of the device 100. In some embodiments, the one or multiple force transmission system(s) 120 may be configured to transmit the force and motion between the flywheel system 210 and the hand H. In some embodiments, one or multiple intermediary linear and/or rotational motion mechanisms may be configured to translate a rotational motion of a subjects hand, due to tremor, to, to a rotational motion of the flywheel 210, and vice versa. In some embodiments, the device 100 may comprise a first force transfer system 120a configured to transmit the force and motion the hand H to a spring system 220. The device 100 may further comprise a second force transfer system 120b be configured to transmit the force and motion from the spring system 220 to the flywheel 210. FIG. 11A1 illustrates an exemplary flow diagram for the force and motion being transmitted from the hand H to the flywheel 210.

In some embodiments, a force transmission system 120 may comprise a cable-driven joint 300 configured to carry and transmit the hand tremor motion. The cable-driven joint 300 may comprise one or multiple attachment pieces 310 on the hand. In some embodiments, the one or multiple attachment pieces 310 comprise one or multiple attachment points 305 configured for one or multiple cables 315 to be attached. In the exemplary embodiment illustrated in FIG. 2A1 the attachment piece 310 may comprise “rigid knuckles” configured to be rested between one or multiple fingers of a subject's hand H. The rigid knuckles 310 may comprise one or multiple screws or tensioners configured to increase and/or decrease a tension of cables 315 affixed to the attachment points 305. In some embodiments, a first end of the cable 315 may be configured to be fixedly attached to one or more of the attachment points 305. In some embodiments, the cable-driven joint 300 may comprise a first top component and a second bottom component. The top component may comprise a first attachment point 305a and a first set of one or multiple pulleys 320a. The bottom component may comprise a second set of one or multiple pulleys 320b. The second component may further comprise a second attachment point 305b. In some embodiments, cable 315 may be configured to be wrapped around the one or multiple pulleys 320a that may be affixed to a wearable base (i.e., an upper wearable base 110a) or housing of the device 100. The device 100 may further comprise a bottom wearable base 110b. The one or multiple pulleys 320b may be configured to be affixed to the bottom wearable base 110b. The cable 315 may be configured to further wrap around the one or multiple pulleys 320b positioned on the bottom wearable base 110b. The other end of the cable 315 may then be affixed to the same attachment point 305a or another attachment point 305b on the attachment piece 310. In some embodiments, there may be multiple attachment pieces. In some embodiments, the multiple attachment pieces may be attached in some capacity to each other. In some embodiments, the cable 315 may be partially wrapped, fully wrapped, or wrapped multiple times around the one or multiple pulleys 320. In some embodiments, the cable 315 may wrap around the one or multiple pulleys 320a on the first wearable base 110a alone. The cable 315 may be inflexible. In some embodiments, the inflexible cable may be configured such that it does not stretch or compress, or does so minimally when under tension or compression. The cable may be in tension in the setup. The cable tension may be controlled manually, semi-automatically or automatically.

In some embodiments, the first force transmission system 120a may be configured to decrease a distance between the first cable attachment point 305a and a first cable-wrapped pulley 320a on the top wearable base 110a in response to an upwards motion of a subject's hand H during a tremor. The decrease in distance may increase the distance between the second cable attachment point 305b and the final cable-wrapped pulley 320b. This may result in the movement of the cable 315. With sufficient tension in the cable 315, the friction between the cable 315 and the pulleys 320 it is wrapped around may be high enough that the resulting movement is that the cable 315 and pulleys 320 move together. As shown in FIGS. 2A1-2A5, the cable 315 may be pulled toward the bottom attachment point 305b and the pulleys 320 may rotate clockwise or counterclockwise, depending on the way the cable 315 is wrapped around the pulleys 320. In some embodiments, the first force transmission system 120a may be configured to translate the rotational motion of the hand H due to tremor to a rotational motion of a rotational mechanism (i.e., pulleys 320) in the first force transfer system 120a in the device 100, (R2R mechanism).

In some further embodiments, the device 100 may further comprise an intermediary or first force transmission mechanism configured to convert a rotational motion and force of a rotational mechanism and/or a subject's hand H to linear motion, (R2L mechanisms) The intermediary force transmission mechanism may be configured to be attached to the cable-driven joint mechanism 300. In some embodiments, one or multiple gears 330 may be fixedly attached to one or multiple pulleys 320 such that they may rotate together. The gear 330 may be configured to be meshed with a first rack 345 in a rack and pinion system 340 such that the rotational motion of the gear(s) 330 translated to the linear motion of the rack 345. The first rack and pinion system 340 may be attached to a second rack and pinion system 440 via a spring system 220. In some embodiments, device 100, may further comprise a second force transfer system 120b configured to transmit the motion and force from the spring system 220 to a flywheel system 210. The second force transfer system 120b may comprise the second rack and pinion system 440. The second force transfer system 120b may further comprise a mechanical advantage system 400. The spring system 220 may be configured to exert a force on the second rack and pinion system 440. In some embodiments, the mechanical advantage system 400 may comprise a pinion 245 fixed about its center and attached to a gear 240 in the flywheel-mechanical advantage system 400. The pinion 245 and the gear 240 may be configured to displace the flywheel 210 rotationally, and not linearly. In a non-limiting example, the force exerted by the spring system 220 on the rack and pinion system 440 may linearly displace the rack 445. The linear displacement of the rack 455 may translate to the angular displacement of the pinion 245, which may also translate to the angular displacement of the gear 240. The angular displacement of the gear 240 may translate to an angular displacement of the pinion 215 and the flywheel 210. In this way, the motion of and force exerted by the hand H due to the tremor may be transmitted to the flywheel 210 and back through one or multiple intermediary mechanisms and/or secondary force transmission systems. In some embodiments, guide rails may enable and/or support the movement of force transmissions systems such as the rack and pinions systems. Non-limiting examples include ball bearing carriages, linear shaft rails, V-groove rails, other profile guide rails, telescoping rods, scissor mechanism, slide guides, linear bearings with shafts, linear guide shafts, air bearings, and magnetic bearings.

In some embodiments, a cable-driven joint 300 as described herein, may further comprise at least one tensioner configured to maintain, increase, and/or decrease the tension of the cable 315. Users may have different hand H and wrist WR sizes, and a tensioner may be configured to ensure that there is enough tension in the cable 315 for smooth operation of the cable-driven joint 300 regardless of a user's hand H and wrist WR size. Too much slack in the cable 315 may result in the cable 315 sliding relative to the pulleys 320 and/or the pulleys 320 not rotating. For instance, threaded screws 305 and/or bolts may be used increase or decrease cable 315 tension. Tightening the screw 305 may increase cable 315 and loosening the screw 305 may decrease cable 315 tension. In some embodiments, the tensioners may be manually adjusted by the user. In some embodiments, the tensioners may be controlled automatically or semi-automatically with the aid of motors or actuators. Spring-loaded tensioners may also be used, in which spring(s), pre-loaded or not, may exert forces on the cable 315 to make it taut. The spring pre-load may be adjusted by the user to increase or decrease the cable 315 tension. Pivot point mechanisms may also be employed as to control cable 315 tension. The cable 315 may additionally be wrapped around pivots such as idler pulleys, idler gears, or capstans on the wearable base 110. These pivots may move linearly on the wearable base and their position may be controlled by the user via intermediary mechanisms. In some embodiments, the pivots are configured to increase and/or decrease the tension of the cable 315 based on the direction that the pivots are moved or displaced. In some embodiments, the pivot points may be configured to rotate to pick up the cable 315 slack. In some embodiments, other tension control elements may be used to control the cable 315 tension, including but not limited to turnbuckles, winches, clamps, and weighted tensioners. In some embodiments, the pivot locations may be manually adjusted by the user. In some embodiments, the pivot locations may be controlled automatically or semi-automatically with the aid of motors or actuators.

In some embodiments, the FSD system 200, may comprise a ratchet and pawl mechanism configured to maintain and/or change the tension in the wire of the cable-driven joint mechanism. The ratchet and pawl mechanism configured to maintain and/or change the tension in the wire of the cable-driven joint mechanism may be configured to function the same or similarly a ratchet and pawl mechanism used to restrict spring coil deflection, as described herein.

In some embodiments, a device 100 as described herein may comprise a bottom wearable base 110b. The bottom wearable base 110b may further comprise one or more additional FDS system(s) configured to facilitating a cable-driven joint 300 or other first force transfer systems 120. The additional FSD system(s) may improve the overall ability device to reduce the amplitude of the hand tremor.

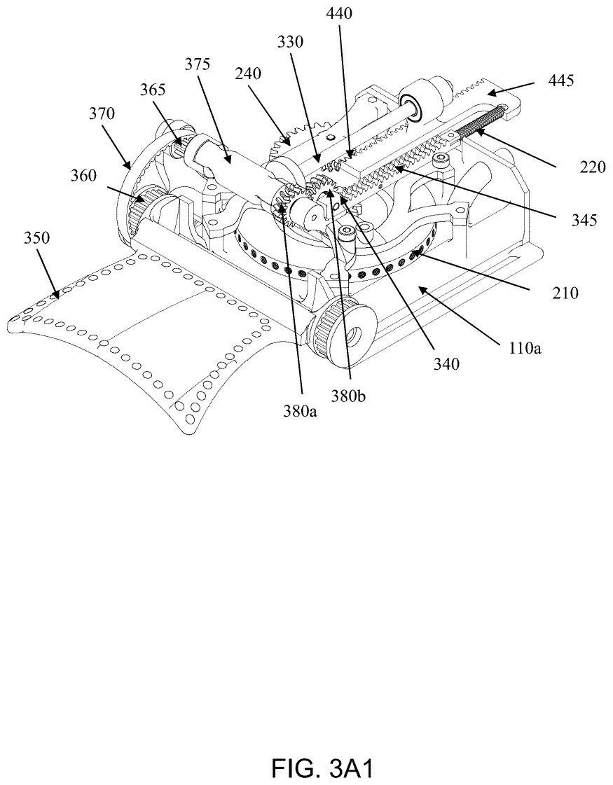

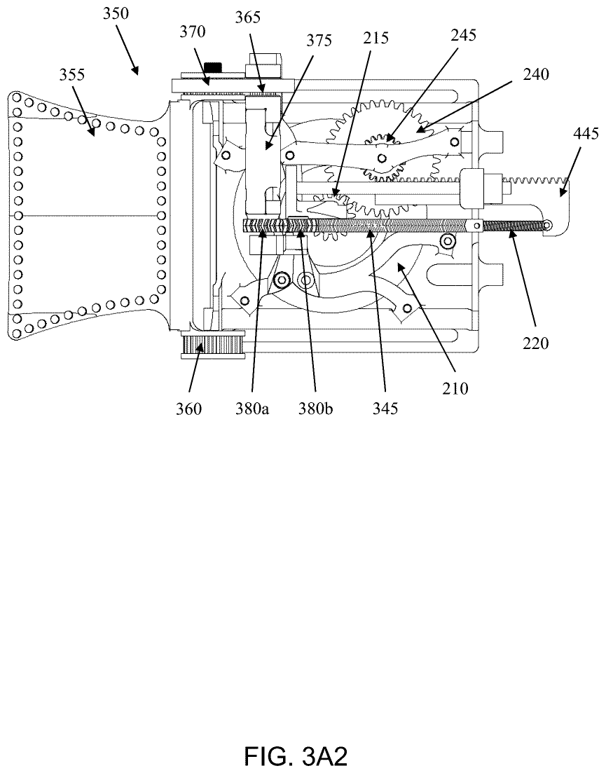

In some embodiments, a first force transfer system 120a that carries and transmits a hand tremor motion may comprise one or multiple belt and pulley systems 350. Referring to FIGS. 3A1-3A2, one or multiple attachment pieces 355 may be fixedly attached to the hand H such that they are configured to rotate with the hand H. The attachment piece(s) 355 may be configured to be attached to the hand H with a glove or band. The glove or band may be configured to cover at least some part of the attachment piece 355 and wrap around at least some portion of the hand H and/or wrist WR and/or distal forearm FA. The attachment piece(s) 355 may comprise at least two separate attachment pieces (i.e., a first attachment piece and a second attachment piece) configured to be rigidly attached to each other via mechanisms including but not limited to sliding joints, hinges, threaded rods, bolts and nuts, turnbuckles, ratcheting straps, clamps, and/or magnetic attachments.

The first attachment piece may be aligned with the top of the hand and the second attachment piece may be aligned with the bottom of the hand. This configuration may allow the attachment pieces to exert forces on both sides of the hand without using a glove or band. The attachment pieces 355 may comprise at least one rigid element with one or multiple pulleys 360. The attachment piece may be attached to the wearable base 110 via these pulleys 360. The pulleys 360 may be attached such that they are free to rotate about their rotation point (e.g., their centers). The pulley 360 rotation axis may be aligned with the center of the user wrist WR. The pulleys 360 may be connected to at least one secondary pulley 365, supported by a wearable base 110a of the device. The pulleys 360 may be connected to the at least one secondary pulley with at least one belt 370. In some embodiments, the pulley-belt system may be configured to translate the rotational motion of the hand H to rotational motion of a rotational mechanism in the FSD device. The rotational motion may then be translated to a linear motion via one or multiple intermediary force transmission mechanisms configured to translate rotational motion and force to linear motion and force (i.e., rack and pinion systems 340). In some embodiments, the intermediary force transmission mechanism(s) may further include, but are not limited to, scotch-yoke, slider-crank, lead screw, CAM-follower, cylindrical-CAM mechanisms, or a combination thereof.

Referring to the exemplary embodiments illustrated in FIGS. 3A1-3A2, an upwards motion of the hand H during a tremor may rotate the pulley 360 on the attachment piece 350 clockwise. The rotation of the pulley 360 may be configured to rotate the at least one other secondary pulley 365 clockwise as well via a belt 370 (pulley-belt system 350). The secondary pulley may be rigidly attached to at least one gear 380a, which may also rotate clockwise. This gear 380a may be attached to a first rack and pinion system 340 directly, via other gears 380b, or via a gear train to translate the rotational motion to linear motion. The first rack and pinion system 340 may be attached to a second rack and pinion system 440 via the spring system 220. The second rack and pinion system 440 may be configured to cause the flywheel 210 to rotate directly or indirectly via the pinion 245, gear 240, and pinion 215 in the flywheel-mechanical advantage system 400. In this way, the motion of the hand tremor may be translated to the (phase shifted) motion of the flywheel 210, and vice versa.

In some embodiments, the motion of the hand H due to tremor may be transmitted directly to the flywheel system via at least one R2R or R2L mechanism. In some embodiments, the motion of the hand H due to tremor may be transmitted to the flywheel system 210 via one or multiple intermediary R2R and/or R2L mechanisms.

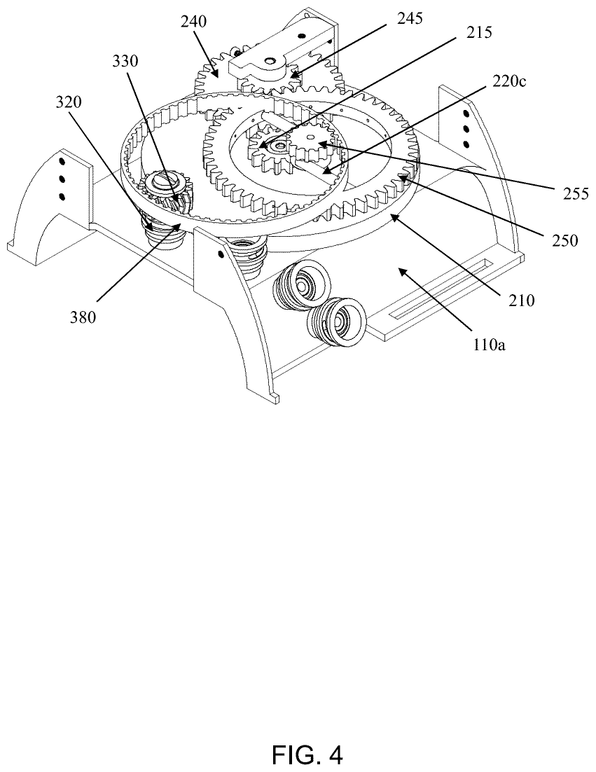

Referring to the exemplary embodiment illustrated in FIG. 4, a first force transfer mechanism 120a may comprise a cable-driven joint mechanism. Second pulley 330 be may fixedly attached to a first pulley 320, position on the wearable base 110 (i.e., 110a or 110b), and configured to rotate with the first pulley 320. The second pulley 330 may be connected to an intermediary force transmission system. The intermediary force transmission system may comprise a belt-pulley mechanism. The belt-pulley mechanism may comprise a third pulley 255 and a belt 380. The second pulley may be connected to a third pulley 255 by the belt 380. The third pulley 255 may be attached to an outer gear 250 by a spring system 220. The spring system may comprise one or multiple spring elements. In some embodiments, the one or multiple spring elements comprise at least one of torsional springs 220c and/or arc springs. In some embodiments, compression or extension springs may be used. The outer gear 250 may be meshed to the gear 240 that is part of the flywheel-mechanical advantage system 400, whose rotation will cause the flywheel 210 to rotate as well. The motion upwards of the hand H during a tremor may rotate the pulleys 320 on the wearable base 110. This would rotate the second pulley 330 that is fixedly attached to the first pulley 320. This would rotate the third pulley 255, which is connected to the second pulley 330 via a belt 380. This rotational motion is then transmitted to the rotational motion of the outer gear 250 via torsional springs 220c. The outer gear 250 rotation causes the gear 240 in the mechanical advantage system to rotate as well, as the two are interlocked. This, as a result, causes the pinion 215 and flywheel 210 to rotate. In this way, the rotational motion of the hand H due to tremor is translated to the rotational motion of the flywheel system 210 via intermediary R2R mechanisms.

In some embodiments, a first force transfer system 120 may comprise a slider-crank mechanism. In a non-limiting example, a slider-crank mechanism may be used in place of a pulley-belt system that is illustrated in FIGS. 3A1-3A2. In some embodiments, the slider-crank mechanism may comprise an attachment piece 350. The attachment piece 350 may comprise at least one rigid element and one or multiple cranks instead of pulleys 320. One or multiple sliders or pistons may be connected to a crank of the slider-crank mechanism. The one or multiple sliders or pistons may be configured to be connected directly to the crank. In some embodiments, one or more connecting rods may be configured to directly connect the one or multiple sliders or pistons to the crank. The slider(s) may be attached directly to the spring system 220. The spring system 220 may be attached to the flywheel 210. The spring system 220 may be configured to be directly or indirectly attached to the flywheel 210. In some embodiments, the spring system 220 may be configured to be attached to the flywheel 210 by a rack and pinion system 440. The slider(s) may also first be fixedly attached to one or multiple “wall” pieces. The wall piece(s) may be attached to the spring system 220, which may then be attached to the flywheel 210 either directly or indirectly through intermediaries such as a rack and pinion system 440. In some embodiments, a scotch-yoke mechanism may be used in place of the slider-crank mechanism.

In some embodiments, a first force transfer system 120a may comprise, one or multiple linkage mechanism(s) (i.e., “scissors” linkage mechanisms). The “scissors” linkage mechanism(s) may comprise a first linkage piece configured to be affixed at one or multiple points to the hand H. The first linkage piece may be configured such that its motion is directly linked to the motion of the hand H. In some embodiment, an upward rotation of the hand H may result in the upward motion of the first linkage piece. In some embodiments, the linkage mechanism may be configured such that the upwards movement of the first linkage may cause motion of one or multiple other linkages in the linkage mechanism causing the length of the scissors linkage mechanism is shortened. In another embodiments the linkage mechanism(s) may be configured such that the length of the scissors linkage may be increased due to upward hand H rotation. In some embodiments, the linkage(s) of the linkage mechanism(s) farthest from the hand H may be attached directly to the spring system 220. The spring system 220 may be attached to the flywheel 210. The spring system 220 may be directly or indirectly attached to the flywheel 210. In a non-limiting example, a rack and pinion system 440 is configured to attach the spring system 220 to the flywheel 210. In some embodiments farthest linkage(s) may first be fixedly attached to one or multiple “wall” pieces. The “wall” piece(s) may be attached to the spring system 220. The spring system 220 may then be attached to the flywheel 210. The spring system 220 may be directly or indirectly attached to the flywheel 210. In a non-limiting example, a rack and pinion system 440 is configured to attach the spring system 220 to the flywheel 210. In some embodiments, telescoping rods may be used in place of the scissors linkage mechanism.

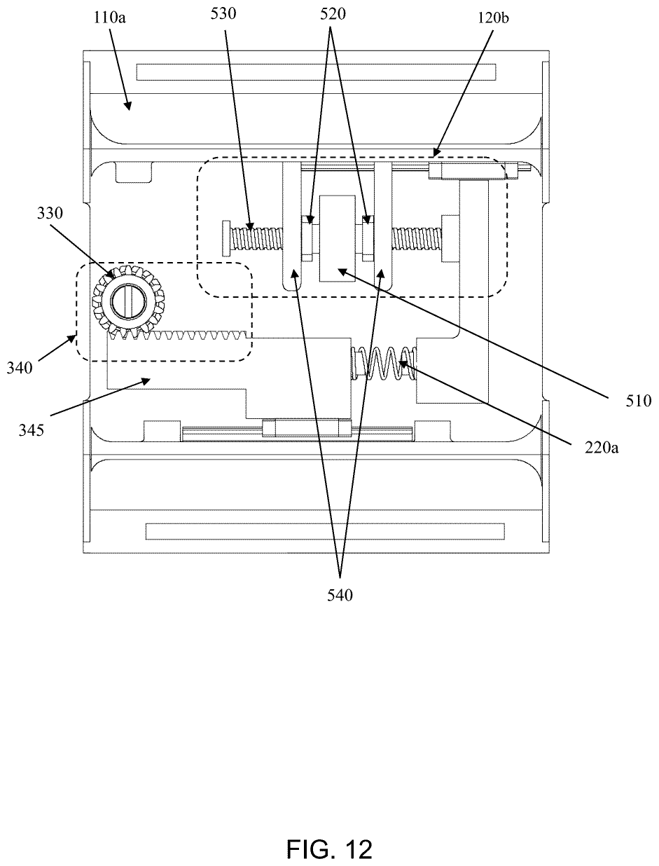

In some embodiments, a second force transfer system 120b, as described herein, may comprise a ball-screw system. Referring to FIG. 12, the ball-screw system may comprise at least one threaded rod 530 and/or screw, at least one nut 520 that may engage with the threaded rod 530, and ball bearings to facilitate the relative motion between the threaded rod 530 and nut 520. The threaded rod 530 may be configured to linearly displace through the nut 520. The nut 520 may be configured to be fixed in place such that it may rotate in place, but not displace linearly. In some embodiments, the threaded rod 530 may have helical grooves along it. The nut 520 may have internal threads that match the threads of the threaded rod 530. One or multiple ball-screw systems may be used. The one or multiple ball screw systems may be used in parallel or in series. In some embodiments, the ball-screw system may further comprise a casing 540 to support, provide stability, and/or alignment the nut 520 and/or threaded screw 530 in the wearable base 110a. In some embodiments, the casing 540 may prevent the nut 520 from displacing linearly. Alternatively, or in combination, thrust bearings may be used to prevent the nut 520 from displacing linearly along the threaded rod 530. In some embodiments, at least one side of the casing 540 may be rigidly attached to one end of the threaded rod 530 and be moveable. The nut 520 may be rigidly attached to the flywheel 510. In some embodiments, the flywheel 510 may be press-fit to the outside of the nut 520. In some embodiments, deflection of the hand H due to a tremor may directly or indirectly exert a force on the spring system 220 via a rack and pinion system 340. The spring system 220 may then directly or indirectly exert a force on the threaded rod 530. The force exerted on the threaded rod 530 by the spring system 220 may cause the threaded rod 530 to displace linearly through the nut 520. The linear displacement of the threaded rod 530 through the nut 520 may cause the nut 520, and thus also the flywheel 510, to rotate. In this way, the second force transfer system 120b may transmit motion to the flywheel. The out-of-phase rotation of the flywheel 510 may provide the resisting force to the rotation or deflection of the hand H. In some embodiments, a roller screw or lead screw system may be used in place of the ball screw system. In some embodiments, the second force transmission system 120b may be lubricated to reduce friction and wear. The configuration of the ball-screw system may affect the flywheel 510 effective mass. The ball-screw system may amplify the motion of the flywheel 510 and increase the flywheel 510 effective mass. The flywheel 510 rest mass, flywheel 510 radius, nut 520 mass, nut 520 radius, lead of the screw or threaded rod 530, and threaded rod 530 mass may affect the effective mass of the flywheel 510. Changing these parameters may change the effective mass of the flywheel 510.

One or multiple intermediary R2R and/or R2L mechanisms may be employed in insolation or in combination with one another in transmitting the motion of and force exerted by the hand H to the flywheel 210, and vice versa.

### Biased Contact Force Transmission Mechanism

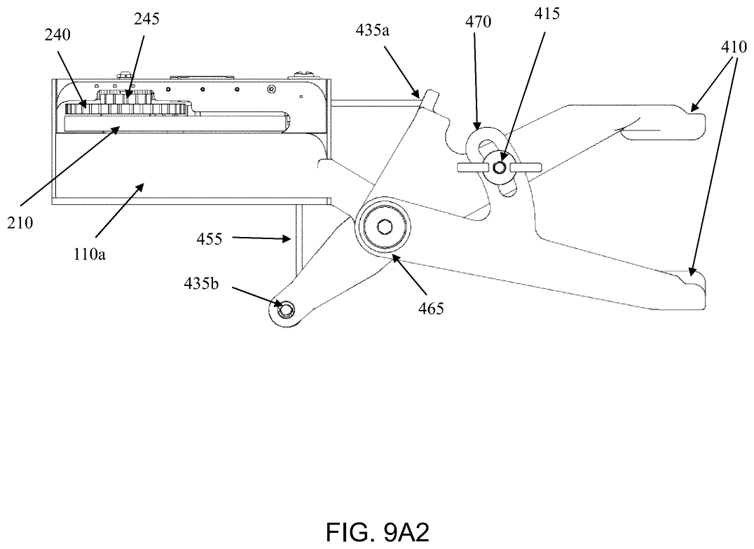

In some embodiments, a force transmission system 120 may comprise a contact mechanism (e.g., an attachment piece 310) configured to be attach to the hand H. In some embodiments, contact mechanism configured to be attach to the hand H may comprise a biased contact mechanism. In some embodiments, the biased contact mechanism may comprise a cable-driven joint mechanism may attach to the hand H with one or multiple rigid pieces that extend over at least a portion above and/or a portion below the hand H. Referring to FIGS. 9A1-9A2, the cable-driven joint mechanism may comprise two lever arms 410, one above the hand H and the other below the hand H. The two lever arms 410 may share a pivot point 465 located at or near the center of the wrist WR axis. The two lever arms 410 may be biased against one another, in that there may exist a force aimed at minimizing the angle between the arms 410, such that they are rigidly pressed against the top and bottom of the hand H. In some embodiments, said biasing may be achieved by connecting the two lever arms 410 via a cable 455 with at least sufficient tension to maintain the arms' 410 position against the hand H. The attachment points of the cable 455 on the two arms 410 and the path followed by the cable 445 may be configured such that increasing the cable 455 tension increases the biasing force pushing the lever arms 410 closer together. Increasing cable 455 tension may increase the rigidity with which the lever arms 410 are affixed to the hand H. In some embodiments the device 100 may comprise additional biasing mechanisms to maintain the relative distance or angle between the lever arms 410. The additional biasing mechanisms may include but are not limited to slider joints, revolute joints, ratchet systems, linkages, adjustable clasps, snap fits, Velcro or hook and loop fasteners, and/or pin buckles. The biasing mechanisms may be configured to vary the relative angle of the rigid pieces (e.g., lever arms 410). The relative angle of the rigid pieces (e.g., lever arms 410) may be configured to be adjusted to accommodate a wide range of users with differently sized hands and/or wrists. As illustrated in the exemplary embodiments of FIGS. 9A1-2A2, the relative angle between the lever arms 410 may be controlled by sliding one arm along the slider joint 470, positioning it against the hand H, and tightening the wing nut 415 to maintain the lever arm positions relative to the hand H. The lever arms 410 may comprise stiff, rigid material. The material stiff rigid material may be configured to better exert force to and from the hand H. In some embodiments, the lever arms 410 may be replaced by other shaped rigids objects. In some embodiments the shaped rigid objects may include but are not limited to plates or rigid meshes. In some embodiments, the biasing force may be adjusted by the user.

In some embodiments, cable 455 may be contained within a cable housing 1025 (i.e., a tube). The tube may guide the cable 455 along an intended path. The tube may comprise a material to provide a low coefficient of friction between the tube and cable (i.e., Teflon). In some embodiments the tube may comprise a low friction lining. This may minimize friction sources. The tube may be positioned along a track on the wearable base 110a. The track may be configured to guide the cable 455 along the device 100. The track may eliminate the need for multiple pulleys 320a to guide the cable 455. In some embodiments, the device comprises a track as described herein and a single pulley 320a to transfer force between the hand H and the FSD system 220. In some embodiments, the user may manually change the path of the cable and/or tube.

In some embodiment, a first end of the cable 455 may be attached to the lever arm 410 positioned on the top of the hand H at a first attachment point 435a. A second end of the cable 455 may be attached to the lever arm 410 positioned on the bottom of the hand H at a second attachment point 435b. The cable 455 may be configured to facilitate a cable-driven joint mechanism, as illustrated in FIGS. 9A1-9A2. In a nonlimiting example, the cable's 455 path from the first cable attachment point 435a to the second cable attachment point 435b, the cable 455 may wrap around the pulley 320a driving the first rack and pinion system 340. The movement of the hand H upwards, for instance, may rotate the lever arms 410 upwards as well. This may be configured to cause the cable 455 to slide along its path and rotate the pulley 320a in one direction (clockwise in FIG. 9A1), which may drive the first rack and pinion system 340. In some embodiments, the cable 455 in the lever-biased mechanism may be used to (i) bias the lever arms 410 to rigidly attach to the hand H and/or (ii) facilitate the cable-driven joint mechanism as described above. FIG. 11A2 illustrates an exemplary flow diagram for the force and motion being transmitted from the hand H to the flywheel 210 of the device 100 comprising a cable-driven joint mechanism.

Referring to FIG. 11A2, in some embodiments, the hand H may deflects as a result of tremor S1101 causing the lever arms 410 deflect with hand H S1102. The lever arms 410 deflection may causes pulley 320a to rotate S1103. The rotation of the pulley 320a may cause a rotation of gear 330 S1104. The gear 330 rotation may cause displacement of a rack 345 S1105. The displacement of the rack 345 may cause a spring 220a to deflect S1106. The deflection of the spring 220a may cause a rack 445 to be displaced S1107. The displacement of the rack 445 may cause pinion 245 to rotate S1108. The rotation of the pinion 245 may cause gear 240 to rotate S1109. The rotation of gear 240 may cause pinion 215 to rotate S1110. The rotation of pinion 215 may cause a rotation of flywheel 210, which may interfere with and reduce the amplitude of the hand tremor.

In some embodiments, one or multiple a contact mechanism configured to be attach to the hand H may be used in places of rigid contact mechanism described above. In some embodiments the one or multiple contact mechanisms may be used in combination.

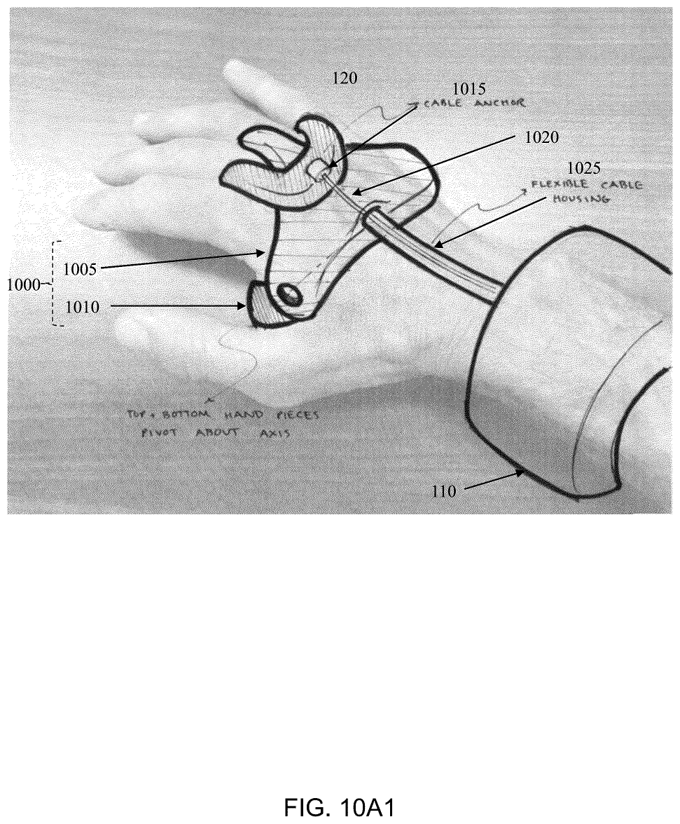

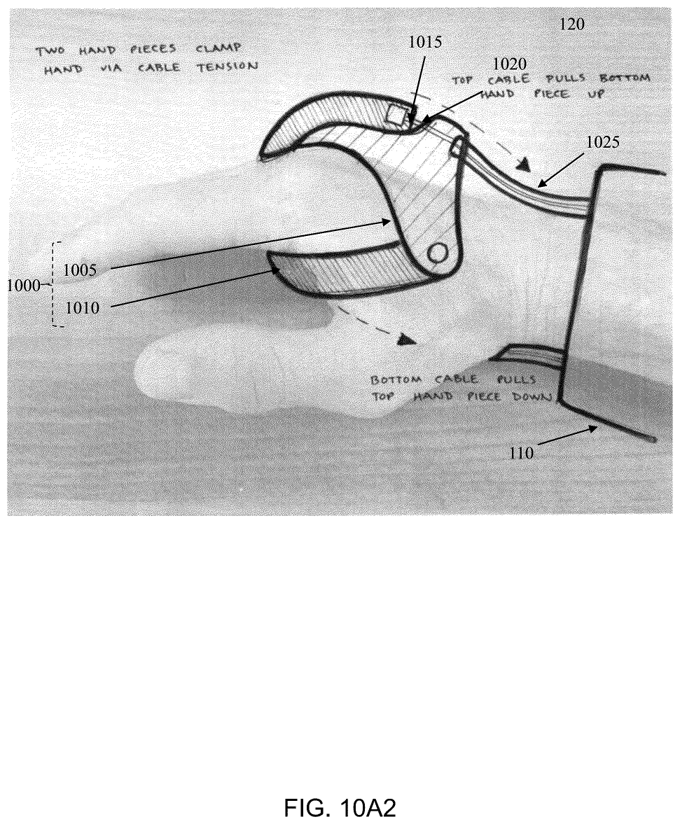

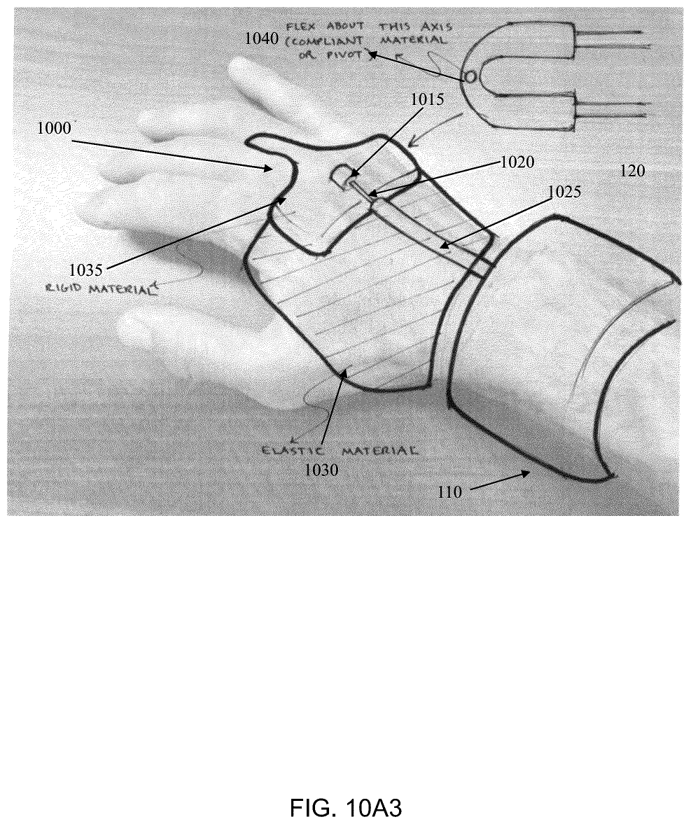

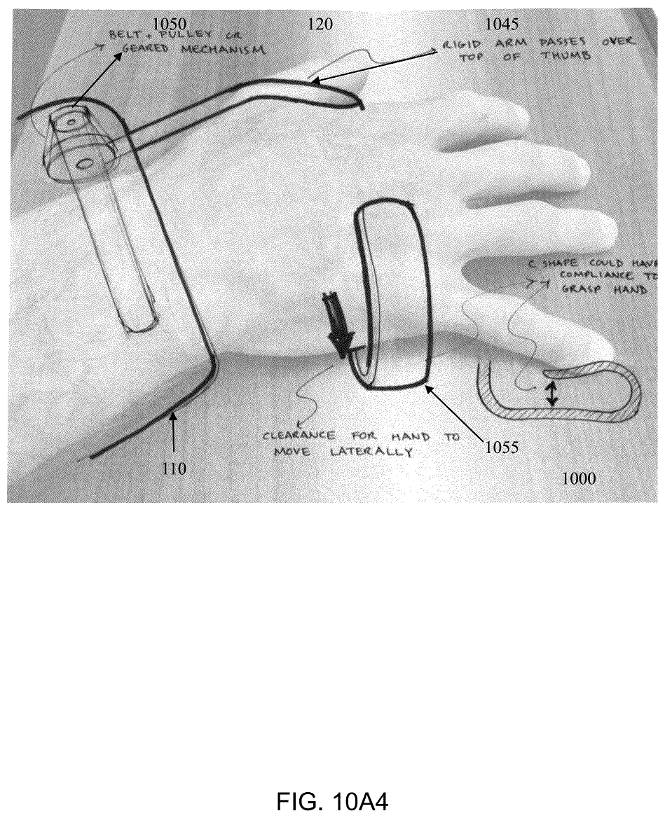

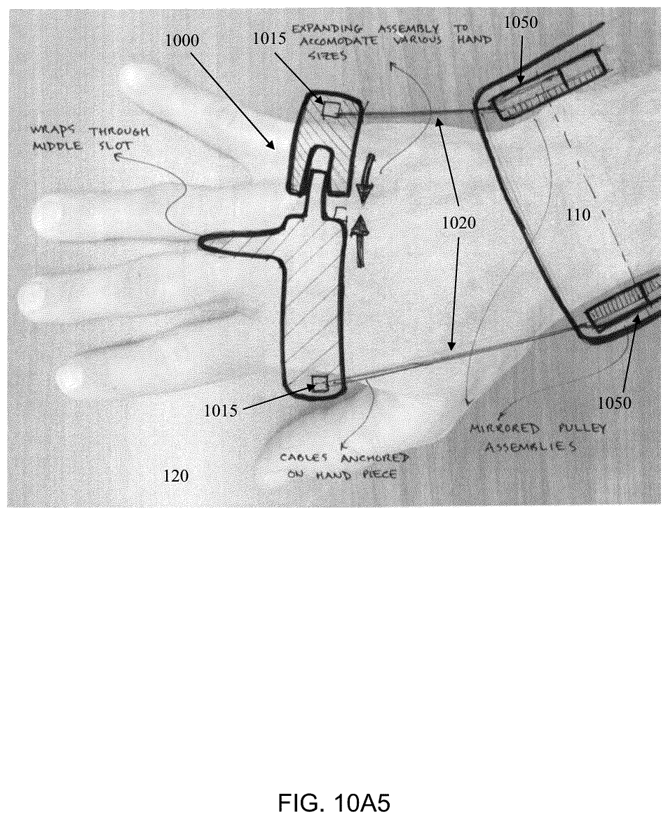

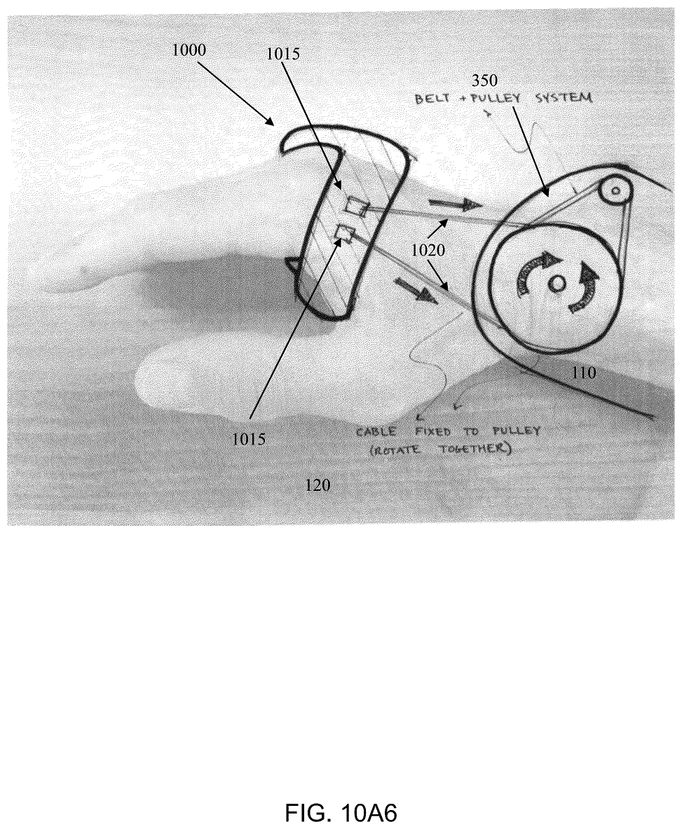

In some embodiments, the biased contact mechanism may comprise contact mechanism 1000 that use one or multiple fingers of the user as anchors to secure and hold the mechanism against the hand. Referring to FIG. 10A1-10A2, one piece of a biased contact mechanism 1000 may be configured to be on top of the hand (top piece 1005) and the other piece may be configured to be on the bottom of the hand (bottom piece 1010). At least part of the top piece may extend to the bottom of the hand. At least part of the bottom piece may extend to the top of the hand. The pieces may be configured to at least partly overlap or overlay each other. The pieces may wrap in between and/or one or multiple fingers of the user. In some embodiments, the pieces be secured around the hand with straps. The bottom piece may be configured to attach to the top piece at the top of the hand. The top piece may be configured to attach to the bottom piece at the bottom of the hand. The pieces may at least partly conform to or be shaped to fit the user's hand. The pieces may be rigid. In some embodiments, at least part of the pieces may be flexible.

Each piece may have attachment or anchor points 1015 where a cable 1020 may be fixedly attached. The cable anchor point 1015 of the top piece may be at the bottom of the hand (“bottom anchor point”). The cable anchor point 1015 of the bottom piece may be at the top of the hand (“top anchor point”). A cable 1020 may be attached to the top anchor point 1015 on one end and to the bottom anchor point 1015 on the other end. In some embodiments, the cable 1020 may be used as part of a force transmission system 120 such as a cable-driven joint. The part of the cable 1020 passing over the top of the hand may pull the bottom piece 1010 up. The part of the cable 1020 passing over the bottom of the hand may pull the top piece 1005 down. There may be sufficient tension in the cable 1020 such that the two pieces 1005, 1010 are rigidly attached to or clamped to the hand. Alternatively, or in combination, the cable tension may enable the hand to deliver and experience forces from both ends. The cable 1020 may be inelastic such that tension in the cable may not extend, or may minimally extend, the length of the cable 1020. The cable tension may be adjusted based on the user hand and wrist size. In some embodiments, the cable may at least partly be contained in a housing 1025.

In some embodiments, the biased contact mechanism may comprise contact mechanisms 1000 that are flexible 1030 in some parts and rigid 1035 in other parts. Referring to FIG. 10A3, a flexible, elastic material 1030 may wrap around at least part of the user's hand. The elastic material 1030 may lie beneath the rigid component(s) 1035. The elastic material 1030 may comprise a breathable, comfortable fabric. The elastic material 1030 may comprise a glove or partial glove. The rigid component 1035 may overlay the elastic material 1030 and may include a pivot 1040 that may at least partly flex about an axis. The rigid component 1035 may wrap around and/or use as anchors the user's hand, fingers, and/or knuckles. The rigid component 1035 may include cable attachment points 1015 above and below the hand. There may be sufficient tension in the cable 1020 such that the rigid component 1035 is rigidly attached to the hand. In some embodiments, the cable 1020 may be used as part of a force transmission system 120 such as a cable-driven joint. The cable tension may be adjusted based on the user hand and wrist size.

In some embodiments, C-shaped contact mechanism 1000 may configured to be used as part of the primary force transmission system 120a. Referring to FIG. 10A4, a C-shaped, rigid piece 1055 may wrap around the hand. In some embodiments, the C-shaped piece may have some compliance such that it may at least partly flex for users to slide their hand in and then grasp the hand. The C-shaped piece may be attached to the wearable base 110 via one or multiple rigid lever arms 1045, cables, or a combination hereof. The lever arm 1045 may at least partly wrap around, or use as an anchor for stability, one or multiple fingers such as the thumb. The lever arm 1045 may be part of the C-shaped piece 1055. The lever arm may have a pivot point around the center of the user's wrist. The C-shaped piece may comprise a pulley or gear 1050 at the pivot point. The pulley 1050 may be used as a part of a pulley-belt system to further transmit the force of motion of the hand to the device components. Alternatively, or in combination, other primary force transmissions systems 120a may be used in conjunction with the C-shaped piece 1055. In some embodiments, the pulley 1050 may be replaced with a gear that transmits motion to the device components via a rack and pinion system. The compliance of the C-shaped piece 1055 may enable its use for users with different sized hands.

In some embodiments, the cables 1015 may be attached to a contact mechanism 1000 on the sides of the hand. Referring to FIG. 10A5-10A6, contact mechanisms 1000 may wrap around the hand and be attached to each other via one or multiple joints. Non-limiting examples include but are not limited to, slider joints, snap fits, ratchet-pawl mechanisms, linkages, adjustable clasps, snap fits, Velcro or hook and loop fasteners, pin buckles, buckle clasps, folding clasps, butterfly clasps, and/or magnetic clasps. The distance between the attachment pieces at the joint location may be adjusted based on the size of the user's hands. In some embodiments, the at least one contact mechanism 1000 may be configured to wrap through or in between one or multiple fingers for support and/or stability. In some embodiments, the contact mechanisms may be cofired to use the thumb and/or knuckles as an anchor point. The cable attachment points 1015 may be configured to be positioned at the sides of the contact mechanism(s) 1000. One or multiple cables 1020 may be employed on one or multiple sides of the contact mechanisms 1000. The ends of each cable 1020 may be attached to the same side of the contact mechanism 1000. Each cable may be connected to and may drive a pulley or gear 1050 located about the user's wrist. The rotation of the hand may cause a rotation in the pulley 1050. The pulley(s) or gear(s) 1050 may be part of one or multiple force transmission systems 120 such as pulley-belt systems 350 that may transmit the force and motion from the hand to other components in the device. There may be sufficient tension in the cable 1020 such that the contact mechanisms 1000 are rigidly attached to the hand. The cable tension may enable the rotation the pulley 1050 in response to the hand rotation. The cable 1020 may be inelastic such that tension in the cable 1020 may not extend, or may minimally extend, the length of the cable 1020. The cable tension may be adjusted by the user. In some embodiments, the cables 1020 may be attached to the center of contact mechanisms 1000.

### Second Mechanical Advantage Systems

A second, or secondary, mechanical advantage system may be employed with the one or multiple force transfer systems 120. The second mechanical advantage system may comprise one or multiple mechanisms configured to amplify the motion of the flywheel system 210 given an input displacement and/or force by the hand H. The second mechanical advantage system may be configured to amplify to amplify the motion of the flywheel system 210 given an input displacement and/or force by the hand Hin combination with a flywheel-mechanical advantage system 400. In some embodiments, the second mechanical advantage system may be configured to be an external mechanism attached to a force transfer mechanisms 120. In some embodiments, the second mechanical advantage system may be to be integrated into the force transfer mechanisms 120. The second mechanical advantage system be a mechanical advantage feature that is integrated into the force transmission system 120 and be configured to be manipulate and/or enhance to further amplify the flywheel 210 motion. In some embodiments, amplifying the motion of the hand H as received by the flywheel 210 via the secondary mechanical advantage system may elicit better motion responses from the flywheel 210; small amplitude tremors may not be “felt” by the flywheel 210 due to internal device friction, compliance of components, and/or inertia of device components. The secondary mechanical advantage system may ensure that the hand H movements are “felt” and responded to by the flywheel 210.

In a non-limiting example, a cable-driven joint mechanism 300, as illustrated in FIG. 1. and FIG. 2A1, the cable-driven joint mechanism 300 inherently has an integrated mechanical advantage feature. The integrated mechanical advantage feature's effect may increase by increasing the distance between the pulley 320 on the wearable base 110 and the cable attachment point 305 on the attachment piece 310. Increasing this distance may increase the displacement of the cable 315 and angular displacement of the pulley 320 for a given angular displacement of the hand H. This, in turn, may increase the angular displacement of the flywheel 210 for a given angular displacement of the hand H.

In another non-limiting example, of an integrated mechanical advantage feature for a pulley-belt system, as illustrated in FIGS. 3A1-3A2, the ratio of the diameter of the first pulley 360 (on the attachment piece 350) to the diameter of the second pulley 365 (supported on the wearable base 110) may be configured to be increased to increase the effect of the mechanical advantage. Increasing the ratio of the diameter may increase the angular displacement of the second pulley 365 for a given hand H angular displacement, which in turn may increase the angular displacement of the flywheel 210.

In another non-limiting example, for a linkage mechanism, a greater number of linkages may be used to create a longer mechanism. The movement of each linkage may contribute to the overall displacement of the mechanism. The use of a greater number of linkages may function as an integrated mechanical advantage feature and may increase the overall displacement of the mechanism.

In some embodiments, external mechanical advantage mechanism may comprise one or multiple cycloidal drives, harmonic drives, or involute gearboxes. The cycloidal drives may be employed between the first force transfer system 120 and spring system 220 and/or between the spring system 220 and flywheel-mechanical advantage systems 400. By changing the number of ring pins and/or lobes in the cycloidal disc, the gear ratio and speed of the output shaft may be manipulated, which may influence the extent to which the movement of the flywheel system 210 is amplified. In some embodiments, one or multiple gear trains may be employed. These gear trains may be employed in various locations in the device. For instance, a gear train may be attached to the one or multiple rack and pinion systems to amplify their motion, and therefore amplify the motion of the flywheel system 210 as well. Other mechanisms that may be used as a secondary mechanical advantage system include worm gears, CAM and followers, ball screws, levers, lever-spring systems, Archimedes screw, and/or chain drives.

### Damping Systems

An important parameter that may affect the out-of-phase motion of the flywheel system 210, and thus also the hand H motion, is the damping constant in the device. Changing the value of this parameter may influence the extent to which the hand tremor amplitude is decreased. Increasing this value may decrease the amount by which the tremor amplitude is reduced, while decreasing this value may increase the amount by which the tremor amplitude is reduced, assuming that the out-of-phase motion of the flywheel system 210 is optimally calibrated to the tremor frequency. If the tremor frequency changes, the frequency adjustment systems described earlier may influence and change the (harmonic) out-of-phase motion of the flywheel 210 so that the FSD system 200 may be optimally calibrated to the new tremor frequency. If the out-of-phase motion of the flywheel system 210 is not optimally calibrated to the tremor frequency, it may not be the case that increasing the damping constant may decrease the amount of tremor reduction or that decreasing the damping constant may increase the amount of tremor reduction. This may depend on factors such as the actual value of the damping constant and the extent to which the FSD system 200 is or is not optimally calibrated to the tremor frequency.

As used herein, an FSD system being tuned to a tremor frequency may refer to an FSD configuration in which the relationship between the flywheel effective mass, spring system effective spring constant, and effective damping constant mat be optimized to best counteract tremor at the given frequency. In some embodiments, all else kept equal, an FSD system with a high damping constant may be more effective in reducing tremor amplitude than an FSD system with a low damping constant if the FSD system is not tuned to the tremor frequency. This may depend upon the difference in the values of the damping constant and/or the degree to which the FSD systems are not tuned to the tremor frequency.

Many factors may contribute to the value of the damping constant. The damping constant may be non-zero even if there no specific system or mechanism to modulate the damping in the device. For instance, internal sources of friction in the device and/or air resistance may contribute to the non-zero value of this parameter. Altering the materials used, adding lubrication, and/or adjusting the extent of the contact between components may be used to impact the damping constant. In some embodiments, the value of the damping constant may be controlled and/or changed. One or multiple mechanisms and/or components may be used in isolation or in combination with one another to manipulate this parameter.

One or multiple adjustable dampers may be employed. For instance, at least a section of the FSD system 200, such as the spring system 220, flywheel system 210, and/or force transfer system(s) 120, may be contained in or interact with a chamber filled with fluid. Fluid properties such as the fluid viscosity may be altered to change the damping constant of the system. For instance, electrorheological or magnetorheological fluids may change their viscosity in the presence of an electrical or magnetic field, respectively. By controlling characteristics of these fields, such as their strength, the change in viscosity may be controlled. In non-Newtonian fluids, changing the hydraulic pressure of the fluid may change its viscosity. Changing the fluids temperature may also change its viscosity. Increasing the viscosity of the fluid, for instance may generally increase the damping constant in the system. Decreasing the viscosity of the fluid may generally decrease the damping constant in the system. In some embodiments, changing one or more of these parameters may change the effective spring constant in a hydraulic spring system.

In some embodiments, the friction between systems or components may be changed. For instance, actuators may increase the force or extent of contact between the flywheel 210 and gear 240 axially, radially, or otherwise in the flywheel-mechanical advantage system 400. The greater the force of contact or pressure between the two parts, the greater the frictional force between them as they rotate in opposite directions. The distance or pressure between parts or components may be controlled using springs. Springs may be attached to components on at least one end. One end of the spring may be attached to the wearable base 110a or other components in the device. The degree to which the spring is pre-loaded or deflected may affect the pressure or force between components. In some embodiments, cables and/or ratchet and pawl mechanisms may deflect springs and/or hold springs in certain positions. Motors and/or actuators, pneumatic systems, and hydraulic systems may enable or facilitate such mechanisms. This may increase the damping constant in the system. Similarly, actuators may insert or remove Teflon sheets between components such as the flywheel 210 and gear 240 in the flywheel-mechanical advantage system 400. The coefficient of friction between the Teflon sheets and the components or between multiple sheets of Teflon may be lower than the existing coefficient of friction between the components. This may decrease the damping constant in the system.

In some embodiments, the air damping in the system may be adjusted. For instance, fins and/or baffles may be inserted or reoriented via actuators such that the effect of air resistance in the system is increased. For instance, these fins and/or baffles may be placed and/or reoriented on the flywheel 210, gear 240, and/or second rack and pinion system 440. The reorientation of the fins may involve changing their angle of attack. The angle of attack may influence the extent of air resistance experienced. Increasing the effect of air resistance may increase the damping constant in the system.

In some embodiments, magnetic damping systems may be employed. The magnetic damping system may be employed whether magnetic spring systems are included in the FSD system 200 or not. Electromagnets and/or permanent magnets may be used. In the case of magnetic springs, a coil may be placed around the magnetic spring system such that the movement of the magnets, or components with conductive material or surfaces, through the coil may induce an EMF in the coil; this may generate a new magnetic field that may oppose the existing magnetic field and thus damp the motion of one or more components that may be attached to the magnets (e.g., the racks in the rack and pinion systems). In some embodiments, this may induce eddy currents in the conductive material. This may increase the damping constant of the overall system. In the case of no magnetic springs, one or multiple electromagnets and/or permanent magnets may be positioned in various locations or attached to various components in the device. With electromagnets, the strength and direction of the electric current passing through the coil may be controlled. This may generate time-varying magnetic fields if the electromagnet is placed on moving parts or the current is time-varying. It may produce stable magnetic fields if placed on stationary parts, for instance. The electromagnets may interact with one another and/or with permanent magnets in the system. This interaction may be in the form of attractive or repulsive forces depending on variables such as the magnet(s) polarity and magnetic field strength and orientation. This may determine the magnitude and direction of the forces applied. The current passing through the coils in the electromagnet may be controlled to generate these forces to increase or decrease the damping in the system (e.g., the forces may be scaled based on the rotational speeds of the flywheel 210 and hand H). In some embodiments, the magnetic field strength and/or direction may be changed to influence the behavior of ferrofluids to manipulate the system damping constant. In some embodiments, the magnetic field strength and/or direction may be changed to influence the behavior of ferrofluids to manipulate the system spring constant. The ferrofluids may be placed between or interact with force transmission systems. In some embodiments, they may be used as a fluid in a hydraulic spring system, as disclosed herein. Changing the magnetic field in the presence of the ferrofluids may affect the fluid viscosity, and thus the extent of damping in the system. In some embodiments, applying a magnetic field may increase the viscosity of the ferrofluid. In some embodiments, changing the magnetic field parameters in the presence of the ferrofluids may affect the effective spring constant when used in the context of a hydraulic and/or magnetic spring system.

### Computer Systems

The present disclosure provides computer systems that are programmed to implement methods of the disclosure. FIG. 13 shows a computer system 1301 that is programmed or otherwise con-figured to perform one or more steps of methods described herein and/or control one or more aspects of the systems and methods described herein. The computer system 1301 can regulate various aspects of the present disclosure, such as, for example, spring tension, cable tension, effective mass of the FSD systems 200; recording bio signals of the patient (i.e., mechanical movement and tremor frequency); analyzing recorded signals and/or one or more other inputs; and generating an output based on the recorded signals and signal analysis.

The computer system 1301 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently pro-gram or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1301 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus with-in a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, visual representation of stimulation parameters; graphical representation of recorded response signals, an indication of the non-invasive assessment of the brain state of the person, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1305. The algorithm can, for example, control stimulation protocols, perform data analysis and generate an output based on the data analysis. Software included in the implementation of the FSD systems 200 may be stored in the memory. The software may include but is not limited to firmware, one or more applications, program data, one or more program modules, and other executable instructions. The programmable portion may be configured to retrieve from memory and execute, among other things, instructions related to the controlling, adjusting, and/or calibrating one or more elements or systems of the device 100 as described herein. In a non-limiting example, the programmable portion may be configured to execute instructions retrieved from the memory for adjusting a coil restrictor in response to a change in tremor frequency sensed by one or multiple sensors.

In some embodiments, a first single or multiple-axis IMU may be attached to the wearable base 110a or a component in the force transmission systems such as a lever arm 410 or the rack 345 in the first rack and pinion system 340. A second single or multiple-axis IMU may be attached to the flywheel 210 or the rack 445 in the second rack and pinion system 440.

In some embodiments, the computer system 1301 may comprise a microcontroller. The micro controller may be configured to use the raw data collected from the IMUs to differentiate between the motion of the tremor and other hand motion by the user. The microcontroller may be configured to analyze the difference in data collected between the IMUs to determine the differentiation between the motion of the tremor and other hand motion by the user. The processed data may be used to determine the user's tremor characteristics such as tremor amplitude and frequency. In some embodiments, the characteristics may be calculated and determined in real time.

In some embodiments, the tremor characteristics are calculated in real time and changes in the tremor characteristics may be determined in real time as well. Changes in a user's tremor frequency may reduce the effectiveness of the device because the FSD system 200 may have been configured to best counteract tremor at the previous tremor frequency.

In response to a tremor frequency change, the FSD system 200 configuration may be changed automatically or semi-automatically. The effective mass of the flywheel(s) 210 and/or the effective spring constant of the spring system 220 may be adjusted to reconfigure the FSD system 200 such that it is optimized to best reduce the tremor at the new frequency. Upon determination of the previous and new tremor parameters, the microcontroller may do nothing, change the flywheel 210 effective mass, change the spring system 220 effective spring constant, and/or change the effective damping constant of the FSD system 200 using mechanisms such as those described herein

The microcontroller may also collect and/or store raw and/or processed data of the user's tremor characteristics over a period of time. This information may be relayed to the user via wireless data transmission, such as over Bluetooth or Wi-Fi, to another device such as a smartphone. The information may provide insights regarding a user's tremor characteristics such as frequency and amplitude changes over time. It may also provide insights as to the relation of these tremor characteristics with other parameters such as time of day and activities. This information may be used by the user to manage their tremor as they see fit.

While preferred embodiments of the present disclosure have been shown and described herein, it will be obvious to those skilled in the art that such embodiments are provided by way of ex-ample only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments described herein may be employed in practicing the present disclosure. It is therefore contemplated that the present disclosure shall also cover any such alternatives, modifications, variations, or equivalents. It is intended that the following claims define the scope of the invention and that methods and structures within the scope of these claims and their equivalents be covered thereby.

### Definitions

As used herein, the term “extent” in references to a mechanical advantage mechanism acting on a flywheel may refer to the amount of rotational displacement the flywheel undergoes in response to an input displacement (for instance from the second intermediary force transmission system). The greater the rotational displacement of the flywheel in response to a given input displacement may be referred to as the mechanical advantage mechanism “acting on the flywheel to a greater extent”. The greater extent to which the mechanical advantage mechanism acts on the flywheel, the greater the effective mass of the flywheel.

1. An apparatus for treating tremors in a subject, the apparatus comprising:
a wearable base configured to be worn over at least a part of a joint of the subject;
at least one flywheel-spring-damper (FSD) system comprising:
at least one housing configured to be coupled to the wearable base; and
at least one flywheel configured to counteract tremor movements in the subject, wherein the flywheel-spring damper system is configured to be coupled to the wearable base.

2. The apparatus of claim 1, wherein the wearable base is configured to be worn over at least a joint of an outer extremity of the subject, and wherein the at least one flywheel is configured to counteract tremor movements in the outer extremity of the subject.

3. The apparatus of claim 2, wherein the outer extremity is a hand of the subject, and wherein the wearable base is configured to be worn over a wrist and/or at least a portion of the hand of the subject.

4. The apparatus of claim 1, wherein the FSD system further comprises a spring system comprising at least one spring element configured to be directly or indirectly coupled to the at least one flywheel and the subject.

5. The apparatus of claim 4, wherein the at least one spring element comprises at least a first spring element and a second spring element.

6. The apparatus of claim 4, wherein the at least one spring element comprises one or more coil springs, gas springs, hydraulic springs and/or magnetic springs.

7. The apparatus of claim 1, further comprising a mechanical advantage mechanism configured to be coupled to the at least one flywheel to amplify a rotational motion of the at least one flywheel and increase its effective mass to better counteract a subject's tremor.

8.-10. (canceled)

11. The apparatus of claim 1, further comprising at least one rack and pinion system and/or ball-screw system configured to increase the effective mass of the at least one flywheel.

12.-15. (canceled)

16. The apparatus of claim 7, wherein one or more secondary mechanical advantage mechanisms are configured to be coupled to at least one force transmission system to amplify the tremor movements transmitted by an outer extremity to the at least one flywheel.

17.-19. (canceled)

20. The apparatus of claim 4, wherein the flywheel-spring-damper system further comprises an adjustment element configured to adjust a spring constant of the at least one spring element.

21. The apparatus of claim 20, wherein the adjustment element comprises:
a first adjustment element configured to adjust a spring constant of a first spring element; and
a second adjustment element configured to adjust a spring constant of a second spring element.

22. The apparatus of claim 21, wherein the first and second adjustment elements are configured to restrict or release active spring coils or engage or disengage the at least one spring element.

23. The apparatus of claim 20, wherein the adjustment element is configured to change a gas or fluid pressure of gas or fluid springs.

24.-26. (canceled)

27. The apparatus of claim 7, further comprising a mechanical advantage mechanism configured to change an effective mass of the flywheel by changing an extent of the mechanical advantage.

28. (canceled)

29. The apparatus of claim 7, wherein the mechanical advantage mechanism is configured to adjust a gear ratio or drive ratio of the mechanical advantage mechanism applied to the flywheel.

30.-36. (canceled)

37. The apparatus of claim 1, further comprising a second wearable base configured to be worn over at least part of the joint of the subject different than the first wearable base.

38. The apparatus of claim 37, wherein the second wearable base comprises a second FSD system.

39. The apparatus of claim 1, further comprising a secondary flywheel configured to function in parallel or in series with the at least one flywheel.

40. The apparatus of claim 1, further comprising at least one sensor configured to measure and transmit movement information of the subject;
a software module configured to receive the movement information measured by the at least one sensor and record and analyze the measured movement information, wherein the movement information comprises one or more of frequency, amplitude, or timings of tremors of the subject; and
a controller configured to automatically calibrate one or more settings of the FSD system to optimize damping of the subject's tremor movements based on the analyzed movement information.

41.-48. (canceled)

49. The apparatus of claim 1, wherein a spring constant of the FSD system and/or an effective mass of the flywheel is configured to be manually adjusted by the subject.

50.-55. (canceled)
